(19) United States
(12) Patent Application Publication
(10) Pub. No.: US 2017/0298175 A1
(43) Pub. Date: Oct. 19, 2017

(54) HIGHLY ELASTIC AND MOLDABLE POLYESTER BIOMATERIAL FOR CARDIAC TISSUE ENGINEERING APPLICATIONS

(71) Applicants: Milica Radisic, Toronto (CA); Locke Davenport Huyer, Toronto (CA); Miles Montgomery, Toronto (CA)

(72) Inventors: Milica Radisic, Toronto (CA); Locke Davenport Huyer, Toronto (CA); Miles Montgomery, Toronto (CA)

(21) Appl. No.: 15/487,115

(22) Filed: Apr. 13, 2017

Related U.S. Application Data
(60) Provisional application No. 62/322,671, filed on Apr. 14, 2016.

(51) Int. Cl. C08G 63/668; C08J 3/24; A61L 27/36; C12N 5/00; A61L 27/18; A61L 27/38; G01N 33/50; C12N 5/077

(52) U.S. Cl. CPC C08G 63/668; G01N 33/5061; C08J 3/24; A61L 27/3625; A61L 27/3687; C12N 5/0657; C12N 5/0062; A61L 27/18; A61L 27/3834; A61L 27/3826; A61L 27/3691; C08J 2367/00; C12N 2513/00; C12N 2533/30

(57) ABSTRACT

The present invention provides a new polyester biomaterial through a simple one-step polycondensation synthesis. 124 polymer exhibited highly elastic properties under aqueous conditions that were tunable according to the UV light exposure, monomer composition, and porosity of the cured elastomer. Its elastomeric properties fell within the range of adult heart myocardium, but they could also be optimized for higher elasticity for weaker immature constructs. The polymer showed relatively stable degradation characteristics, both hydrolytically and in a cellular environment, suggesting maintenance of material properties as a scaffold support for potential tissue implants. When assessed for cell interaction, this polymer supported rat cardiac cell attachment in vitro as well as decreased fibrous capsule formation in vivo when compared to poly(L-lactic acid) control. This suggests the potential applicability of this material as an elastomer for cardiac tissue engineered constructs. Furthermore, the highly elastic polyester could be molded and photocrosslinked into a complex mesh structure with feature size on the order of tens of micrometers, demonstrating utility in cardiac tissue engineering constructs.

### CROSS-REFERENCE TO RELATED APPLICATIONS

This patent application claims priority to U.S. Provisional Patent Application Ser. No. 62/322,671, filed on Apr. 14, 2016, titled “A Highly Elastic and Moldable Polyester Biomaterial for Cardiac Tissue Engineering Applications”, and is hereby expressly incorporated by reference as part of the present disclosure.

### BACKGROUND OF THE INVENTION

There is an increasing need for suitable biodegradable polymers for the advancement of tissue engineering. Engineered tissue constructs rely on biomaterial polymers as support structures for tissue construction. These materials form a support mechanism that assist immature groups of cells to develop into complex tissue networks that exhibit the properties of native cells, and support the integration of these complexes into the host surroundings (PCT application No. PCT/CA2014/051046, which is incorporated herein by reference). Recently, the tissue engineering community has increasingly focused on the utilization of polyester biomaterials for scaffold construction. These polymers are desirable for their simple synthesis procedure, hydrolytic degradation properties, and elastomeric characteristics. The FDA has approved synthetic materials called polylactones for such purposes, which include poly(L-lactide) (PLA), poly(glycolide) (PGA), and their copolymers (PLGA); however, these polymers are stiff with poor elastomeric properties, thereby limiting their use in soft tissue engineering applications such as blood vessels and cardiac tissues. Successful application of synthetic materials in tissue engineering requires suitable biodegradable materials to closely match the mechanical properties of the natural tissue, to support the maturation of tissue and to withstand deformations without causing irritation to their surroundings.

Biomaterial-based cardiac tissue engineering solutions rely on optimized material properties including elasticity, degradation rate, and material compatibility which mimic the properties of native cardiac tissue for effective development of tissue constructs. Therefore, an ideal biodegradable elastomer for cardiac tissue engineering should exhibit a relatively low Young's modulus, with high elongation and tensile strength. Those materials that are too stiff can restrict tissue contraction and hence tissue maturation.

Poly(octamethylene maleate (anhydride) citrate) (POMaC), a biodegradable polymer based upon citric acid, maleic anhydride, and 1,8-octanediol, has been developed previously to improve the mechanical properties of synthetic materials for tissue engineering applications (see e.g., U.S. Pat. No. 8,574,311, which is incorporated herein by reference). POMaC features a dual crosslinking mechanism (DCM): strong carbon-carbon crosslinking via ultraviolet (UV) photopolymerization through maleic acid and/or ester bond cross-linking via post-polycondensation through free —OH and —COOH groups of citric acid. Citric acid in POMaC provides four functional groups: three carboxylic acids and one hydroxyl group, which together provide a polymer backbone with four arms through its initial pre-POMaC polymer condensation and/or cross-linking via post-polycondensation. Although POMaC provides improved properties in comparison to the FDA approved synthetic materials, its Young's modulus needs to be further improved for soft tissues engineering especially for cardiac applications. This suggests the need of a new and improved elastomeric material with a relatively low Young's modulus, high elongation and high tensile strength for tissue engineered constructs, and in particular, for cardiac tissue applications.

### SUMMARY OF THE INVENTION

The present invention describes a new biodegradable polymer capable of being UV-cross-linked to form an elastomer and methods of making and using this polymer and resulting elastomer for cardiac tissue engineering.

In one embodiment, the polymer disclosed here is poly(octamethylene maleate (anhydride) 1,2,4-butanetricarboxylate) and which is capable of being UV-cross-linked to form an elastomer.

In another embodiment, the invention relates to a biodegradable polymer made by the following polycondensation reaction scheme involving the polycondensation of the following three reactants or their equivalents:

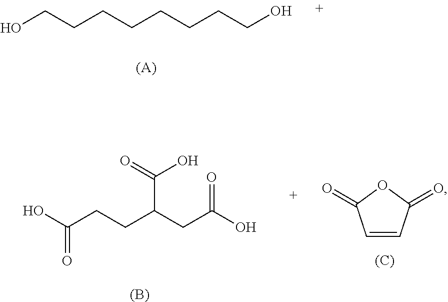

wherein reactant A is at least one diol, e.g., 1,8-octanediol or its equivalents, reactant B is at least one tricarboxylic acid, e.g., 1,2,4-butanetricarboxylic acid or its equivalents, and reactant C is at least one unsaturated di-acid, e.g., maleic anhydride or its equivalents. These three reactants or their equivalents undergo a polycondensation reaction to form the polymer of Formula I:

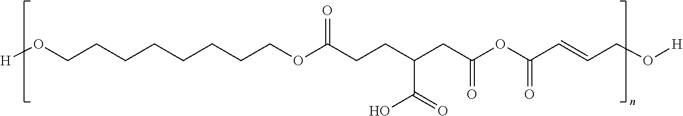

or an equivalent thereof.

By “equivalents” is meant to encompass initial reactants or the resulting polymer and/or elastomer (following UV treatment) formed by a polycondensation of said initial reactants that have substantially the same physical properties, chemical properties, capacity for UV cross-linking, structure, reactivity, and capacity for application as a cardiac elastomeric material. By “substantially the same” is meant, as used herein, said where physical properties, chemical properties, capacity for UV cross-linking, structure, reactivity, and capacity for application as a cardiac elastomeric material are at least 95% as compared to those same features of the polymer of Formula I. Accordingly, this specification is intended to encompass Formula I, but also equivalents of Formula I.

In a preferred embodiment, the present invention describes a new biodegradable polymer capable of being UV-cross-linked to form an elastomer and methods of making and using this polymer and resulting elastomer for cardiac tissue engineering.

In one embodiment, the polymer disclosed here is poly(octamethylene maleate (anhydride) 1,2,4-butanetricarboxylate) and which is capable of being UV-cross-linked.

In another embodiment, the present invention utilizes inexpensive and non-toxic monomers for synthesis of elastomer, such as reactants A, B, and C above, or equivalents thereof.

In one embodiment, the invention provides that a crosslinked elastomer pre-polymer is a polyester formed from monomers of tricarboxylic acid, at least one diol, and at least one unsaturated di-acid through polycondensation reaction.

In another embodiment, the invention provides an elastomer formed by UV-cross-linking a pre-polymer or polyester resulting from a polycondensation reaction of monomers of 1,2,4-butanetricarboxylic acid, at least one diol (e.g., 1,8-octanediol) and at least one unsaturated di-acid (e.g., maleic anhydride).

In some embodiments, these polyesters have relative low viscosity and are suitable for injection through typical needle gauges.

In some embodiments, these polyesters are injected into a mold to form microfabricated structures, and are exposed to ultraviolet (UV) light to form a crosslinked elastomer.

In one embodiment, the invention provides that a crosslinked elastomer pre-polymer is a polyester consisting of monomers of dicarboxylic acid, at least one triol, and at least one unsaturated di-acid through polycondensation reaction. This polyester also has relatively low viscosity for injection through typical needle gauges, and is molded into microfabricated structures and subsequently exposed to UV light to form a crosslinked elastomer.

In another embodiment, an elastomer is featured with a dual-crosslinking mechanism: carbon-carbon crosslinking via ultraviolet (UV) photo-polymerization and ester-bond cros slinking via polycondensation. Utilizing the dual-cros slinking mechanism, the mechanical properties, degradability and functionalities of the polymer can be fine-tuned by controlling the “primary” crosslinking (i.e., the UV crosslinking) and/or the “secondary” crosslinking (i.e., the ester bond crosslinking via polycondensation) to meet the versatile needs of cardiac tissue engineering.

In one embodiment, the invention features a method of fine-tuning an elastomer formed from pre-polymer poly(octamethylene maleate (anhydride) 1,2,4-butanetricarboxylate) by introducing crosslinking.

In some embodiments, the crosslinking can be “intermolecular,” i.e., bonds formed between separate pre-polymers of poly(octamethylene maleate (anhydride) 1,2,4-butanetricarboxylate) or equivalent thereof.

In other embodiments, the crosslinking can be “intramolecular,” i.e., bonds formed between atoms of a single pre-polymer of poly(octamethylene maleate (anhydride) 1,2,4 butanetricarboxylate) or an equivalent thereof.

In some embodiments, the crosslinking can be introduced by UV crosslinking, i.e., “primary” crosslinking. The degree of primary crosslinking can be controlled by adjusting the intensity and/or duration of UV light exposure to the pre-polymer poly(octamethylene maleate (anhydride) 1,2,4 butanetricarboxylate) or an equivalent thereof.

In some other embodiments, the crosslinking can be introduced by chemical reaction, e.g., polycondensation reaction, i.e., “secondary” crosslinking. The degree of secondary crosslinking can be controlled by both temperature and time of the secondary reaction. The optimal amount is dependent on the application, as a higher amount of crosslinking will make a stiffer material. It is best quantified by Young's Modulus, which is directly related to the degree of crosslinking and crosslinking density. In more detail, Young's Modulus (aka elastic modulus) is a mechanical property of linear elastic solid materials. It defines the relationship between stress (force per unit area) and strain (proportional deformation) in a material. Mathematically, Young's Modulus is the ratio of stress (which has units of pressure) to strain (which is dimensionless), and so Young's Modulus has units of pressure. Its SI unit is therefore the pascal (Pa or N/m² or m⁻¹·kg·s⁻²). The practical units used are megapascals (MPa or N/mm²) or gigapascals (GPa or kN/mm²). In United States customary units, it is expressed as pounds (force) per square inch (psi). The abbreviation ksi refers to “kpsi”, or thousands of pounds per square inch. Use of Young's Modulus is well known in the art. In certain embodiments, the invention provides a method comprising:

a. conducting a polycondensation reaction with monomers 1,2,4-butanetricarboxylic acid, at least one diol (e.g., 1,8-octanediol) and at least one unsaturated di-acid (e.g., maleic anhydride) to form a pre-polymer (e.g., poly(octamethylene maleate (anhydride) 1,2,4 butanetricarboxylate));

b. exposing the pre-polymer, optionally having been introduced into a mold, with ultraviolet (UV) radiation for a period of time sufficient to form crosslinking, thereby forming an elastomer. It will be appreciated that the degree or extent of UV crosslinking is in proportion to the time and/or intensity of the UV light administered to the pre-polymer, among other possible factors. Further, it will be appreciated that the degree or extent of elastomeric characteristics will inversely correspond to the degree or extent of crosslinks that are formed between pre-polymer strands. Thus, one may expect to “tune” the elastomer to have increased elastomeric properties by reducing the extent of UV crosslinking, which in turn, can be achieved by reducing the amount of time the pre-polymer is subjected to UV light, or by reducing the intensity of the UV light. Alternatively, one may expect to “tune” the elastomer to be more stiff (i.e., less elastomeric) by increasing the extent of UV crosslinking, which in turn, can be achieved by increasing the amount of time the pre-polymer is subjected to UV light, or by increasing the intensity of the UV light. Tuning can be conducted in accordance with Young's Modulus. In certain embodiments, the invention relates to an elastomer comprising the pre-polymer poly(octamethylene maleate (anhydride) 1,2,4 butanetricarboxylate) which comprises at least 1%, or 2%, or 3%, or 5%, or 7%, or 8%, or 9%, or 10%, or between 5-15%, or between 10-20%, or between 15-30%, or between 25-40%, or between 35-50%, or between 45-60%, or between 55-70% or between 65-80%, or between 75-90%, or up to 100% of all possible primary crosslinks inducible by exposure to UV light.

In a further embodiment, the invention provides a method for inducing up to at least 1%, or 2%, or 3%, or 5%, or 7%, or 8%, or 9%, or 10%, or between 5-15%, or between 10-20%, or between 15-30%, or between 25-40%, or between 35-50%, or between 45-60%, or between 55-70% or between 65-80%, or between 75-90%, or up to 100% of all possible primary crosslinks in the pre-polymer poly(octamethylene maleate (anhydride) 1,2,4 butanetricarboxylate) by exposing the pre-polymer to UV light for a sufficient period of time and at a sufficient intensity per a set quantity and/or concentration of pre-polymers.

In certain other embodiments, the invention relates to an elastomer comprising the pre-polymer poly(octamethylene maleate (anhydride) 1,2,4 butanetricarboxylate) which comprises at least 1%, or 2%, or 3%, or 5%, or 7%, or 8%, or 9%, or 10%, or between 5-15%, or between 10-20%, or between 15-30%, or between 25-40%, or between 35-50%, or between 45-60%, or between 55-70% or between 65-80%, or between 75-90%, or up to 100% of all possible secondary crosslinks inducible by chemical polycondensation reaction.

In a further embodiment, the invention provides a method for inducing up to at least 1%, or 2%, or 3%, or 5%, or 7%, or 8%, or 9%, or 10%, or between 5-15%, or between 10-20%, or between 15-30%, or between 25-40%, or between 35-50%, or between 45-60%, or between 55-70% or between 65-80%, or between 75-90%, or up to 100% of all possible secondary crosslinks in the pre-polymer poly(octamethylene maleate (anhydride) 1,2,4 butanetricarboxylate) by allowing sufficient secondary crosslinks to form by polycondensation reaction.

In another embodiment, carbon-carbon crosslinking in the elastomer can help maintain the wet mechanical properties for the polymers by providing the desired strength and the extent of secondary crosslinking controls the elasticity.

In one embodiment, the invention provides that a crosslinked elastomer is produced from a polyester from monomers of 1,2,4-butanetricarboxylic acid, at least one diol, and at least one unsaturated di-acid through polycondensation reaction; followed by exposing the polyester pre-polymer to UV light to produce a crosslinked elastomer.

In another embodiment, the diol monomer is selected from poly ethylene glycol, saturated aliphatic diols, macrodiols, or a mixture thereof.

In another embodiment, the unsaturated di-acid is selected from maleic acid, fumaric acid, maleic anhydride, fumaryl chloride, or a mixture thereof.

In yet another embodiment, the diol is 1,8-octanediol and the unsaturated di-acid is maleic anhydride.

In another embodiment, the crosslinked elastomer further contains one or more crosslinks.

In another embodiment, the ratio of hydroxyl to carboxylic acid end groups in monomers is 1:1.

In other embodiments, the ratio of hydroxyl to carboxylic acid end groups in monomers is about 1:2, or about 1:3, or about 1:4, or about 1:5, or about 1:6, or about 1:7, or about 1:8, or about 1:9, or about 1:10, or up to about 1:25, or 1:50, or 1:100, or more.

In still other embodiments, the ratio of carboxylic acid end groups to hydroxyls in monomers is about 1:2, or about 1:3, or about 1:4, or about 1:5, or about 1:6, or about 1:7, or about 1:8, or about 1:9, or about 1:10, or up to about 1:25, or 1:50, or 1:100, or more.

In another embodiment, the monomers are stirred at 150° C. to form the polyester through condensation reactions.

In another embodiment, the monomers are stirred at between about 125-135° C., or between about 130-145° C., or between about 140-155° C. , or between about 145-165° C. , or between about 150-175° C., or higher.

In one embodiment, the ratio of tricarboxylic acid and maleic anhydride monomers is 2:3 or 1:4. In still other embodiments, the ratio of tricarboxylic acid and maleic anhydride monomers is about 1:2, or about 1:3, or about 1:4, or about 1:5, or about 1:6, or about 1:7, or about 1:8, or about 1:9, or about 1:10, or up to about 1:25, or 1:50, or 1:100, or more.

In yet another embodiment, the ratio of maleic anhydride monomers to tricarboxylic acid and maleic anhydride monomers is about 1:2, or about 1:3, or about 1:4, or about 1:5, or about 1:6, or about 1:7, or about 1:8, or about 1:9, or about 1:10, or up to about 1:25, or 1:50, or 1:100, or more.

In yet another embodiment, the monomers are stirred for about a minute to several minutes, or for about 5-15 min, or about 10-25 min, or about 20-35 min, or about 30-45 min, or about 40-55 min, or about an hour, or more than hour, such as 1-2, or 2-3, or more hours to form a viscous pre-polymer through condensation reactions. The temperature during stirring may be fixed at a single temperature or it may vary during the course of the polycondensation reaction.

In another embodiment, the crosslinked elastomer is formed by first mixing the polyester pre-polymer with UV initiator, followed by exposing to UV light to form the crosslinked elastomer.

In one embodiment, the crosslinked elastomer formed a scaffold for soft tissue applications.

In another embodiment, the crosslinked elastomer formed a cardiac tissue engineered construct.

In another embodiment, the crosslinked elastomer formed a deformable scaffold element for maturation of human pluripotent stem cell-derived cardiomyocytes.

In yet another embodiment, the crosslinked elastomer formed a deformable scaffold element for in vitro cardiac cell attachment.

In one embodiment, the elastomer of the invention shows favorable elastomeric properties (e.g., lower Young's modulus) which fall in the range of human cardiac tissue.

In another embodiment, the elastomer of the invention can potentially be used as injectable scaffolds for in situ tissue engineering because free radical polymerization is a mild crosslinking process and avoids any harsh processing conditions.

In yet another embodiment, the crosslinked elastomer is formed as a deformable (i.e., bendable) scaffold element for in vitro cardiac cell attachment, and can be used to evaluate the cardiac response to drugs in vitro.

In one embodiment, the invention provides a method of evaluating cardiac response to drugs in vitro using crosslinked elastomer by producing polyester consisting of monomers of 1,2,4-butanetricarboxylic acid; at least one diol; and at least one unsaturated di-acid; injecting polyester into a mold to form micro fabricated structures; exposing the molded polyester to UV to form a crosslinked elastomer; constructing the crosslinked elastomer into a deformable scaffold element; attaching the cardiac cells onto the deformable scaffold element; exposing the cardiac cells with deformable scaffold element to drugs; and monitoring the response of cardiac cells through the deformable scaffold element.

In yet another embodiment, the invention provides a method of engineering a three-dimensional mature cardiac tissue in a bioreactor from single cell types using an elastomer of the invention.

In still a further embodiment, the invention provides a method of engineering any three-dimensional tissue scaffold described in PCT application No. PCT/CA2014/051046, which is incorporated herein by reference, by replacing or substituting the POMaC used therein with an elastomer of the invention, e.g., a crosslinked elastomer of poly(octamethylene maleate (anhydride) 1,2,4 butanetricarboxylate).

In still another embodiment, the invention provides any three-dimensional tissue scaffold described in PCT application No. PCT/CA2014/051046, which comprises or is formed using an elastomer of this invention (e.g., a crosslinked elastomer of poly(octamethylene maleate (anhydride) 1,2,4 butanetricarboxylate)) in place of POMaC used therein, including a three dimensional tissue strand, a three dimensional tissue strand that is suitable for measuring contractile forces, a three-dimensional tissue structure comprising a three-dimensional branched tissue scaffold or matrix having one or more luminal passageways (e.g., mimicking a vascularized three-dimensional tissue structure) integrated therein, and a three dimensional perfusable tissue strand, that is suitable for measuring contractile forces. The scaffold elements of any such three-dimensional tissue scaffolds can be made of an elastomer of the invention (in place of POMaC) and are preferably deflectable, deformable, bendable, or the like, which can be further configured to allow the measurement of contractile forces exerted by the tissue strand on the scaffold elements.

In various embodiments, any three-dimensional tissue scaffold prepared in accordance with the invention or the description of PCT/CA2014/051046 and which comprises an elastomer of the invention (e.g., an elastomer of poly(octamethylene maleate (anhydride) 1,2,4 butanetricarboxylate)) can comprise any suitable cell or can otherwise be formed from any suitable cell, such as cells from cardiac tissue, liver tissue, kidney tissue, cartilage tissue, skin, bone marrow tissue, or combinations of such tissues, or the like. The cells used to grow the three-dimensional tissues can be sourced from anywhere, including from any commercial source, or even sourced from individual subjects or patients. For example, a tissue strand of the invention may be grown starting from a seed of a commercially available hepatic cell line. In another example, a tissue strand of the invention may be grown starting from a seed of cells obtained directly from a subject, e.g., cells isolated from a biopsy. In other embodiments, the three-dimensional tissues of the invention can be grown from a mixture of different cells. Such mixtures of cells can include mixtures of healthy or diseased cells from the same or different tissues, mixtures of cells from different sources or patients, or mixtures of cells from both patients and from commercial sources. The cells used to grow the tissues of the invention can also be genetically engineered cells, such as drug-resistant or drug-sensitive engineered cell lines.

In other embodiments, the cells used to grow the three-dimensional tissues of the invention can be stem cells, including embryonic stem cells (“ESCs”), fetal stem cells (“FSCs”), and adult (or somatic) stem cells (“SSCs”). The stem cells, in terms of potency potential, can be totipotent (a.k.a. omnipotent) (stem cells that can differentiate into embryonic and extra-embryonic cell types), pluripotent stem cells (can differentiate into nearly all cells), multipotent stem cells (can differentiate into a number of cell types), oligopotent stem cells (can differentiate into only a few cell types), or unipotent cells (can produce only one cell type). Stem cells can be obtained commercially, or obtained/isolated directly from patients, or from any other suitable source.

Where applicable or not specifically disclaimed, any one of the embodiments described herein are contemplated to be able to combine with any other one or more embodiments, even though the embodiments are described under different aspects of the invention.

These and other embodiments are disclosed or are obvious from and encompassed by, the following Detailed Description.

### BRIEF DESCRIPTION OF THE DRAWINGS

The above and other features of the present invention will now be described in detail with reference to certain exemplary embodiments thereof illustrated the accompanying drawings which are given herein below by way of illustration only, and thus are not limitative of the present invention, and wherein:

FIGS. 1A, 1B, 1C, and 1D illustrate the polycondensation reaction scheme and NMR characterization of the “124 pre-polymer” (aka poly(octamethylene maleate (anhydride) 1,2,4 butanetricarboxylate)). FIG. 1A depicts the polycondensation reaction. It shows 1,8-octanediol, 1,2,4-butanetricarboxylic acid and maleic anhydride underwent polycondensation (T=150° C.) under a nitrogen atmosphere to generate a viscous 124 pre-polymer fluid. FIG. 1B shows a representative 1H-NMR for the 124 pre-polymer fluid. FIG. 1C illustrates the formation of UV crosslinks to form the 124 polymer, an elastomer. FIG. 1D illustrates the formation of dual sets of crosslinks: UV crosslinks and ester bond formation (due to further polycondensation between adjacent tricarboxylic acid moieties).

FIG. 2A illustrates the assessment of the 124 polymer mechanical properties Mathematical representation of Young's Modulus to “Monomer Ratio (A),” “UV Exposure Energy (B),” and “Poragen Content (C).” The model incorporates the parameters shown in the table above, incorporating statistically significant (p<0.05) main and interaction factors. The 3D heat plot is a graphical representation of the equation of FIG. 2B. FIG. 2C shows a stress strain curve of the 124 polymer compared to POMaC under the same synthesis conditions. The 124 polymer exhibits a more gradual slope, demonstrating more elastic characteristics.

FIGS. 3A, 3B, 3C, and 3D illustrate the mass loss of polymers in solution. FIGS. 3A and 3B. Change in mass of photocrosslinked 124 polymer and POMaC both in their pure form (3A) and with 40% (m/m) initial porogen content (3B) in a transwell atmosphere in contact with neonatal rat cardiomyocytes over a 14 day period at 37° C. (* p<0.05, n=5). FIGS. 3C and 3D. Changes in mass of photocrosslinked 124 polymer and POMaC in pure form (3C) and containing 40% (m/m) initial porogen content (3D) in PBS at 37° C. over a 30 day period (* p<0.05, n=4).

FIGS. 4A, 4B, 4C, and 4D illustrate the change in Young's modulus in solution. FIGS. 4A and 4B. Change in Young's modulus of photocrosslinked 124 polymer and POMaC both in their pure form (4A) and with 40% (m/m) initial porogen content (4B) in a transwell assay with neonatal rat cardiomyocytes over a 14 day period at 37° C. (* p<0.05, n=5). FIGS. 4C and 4D. Changes in Young's modulus of photocrosslinked 124 polymer and POMaC in pure form (4C) and containing 40% (m/m) initial porogen content (4D) in PBS at 37° C. over a 30 day period (* p<0.05, n=5).

FIGS. 5A, 5B, and 5C illustrate that the 124 polymer can be molded into elastic scaffolds which support rat cardiac cell culture. FIG. 5A shows a bright field image of the scaffold at two different magnifications (Scale bar=250 μm (left), 100 μm (right)). FIG. 5B and FIG. 5C. Confocal images of a tissue constructed of 124 polymer scaffold (scale bar=20 μm), where red is F-actin and green is cardiac troponin-T, presenting the ability for cell attachment of rat cardiomyocytes. Magnified images present characteristic cross-striations of cardiac cells.

FIGS. 6A and 6B illustrate the in vitro cytotoxicity assessment of 124 polymer. FIG. 6A. Rat cardiac fibroblast cells seeded on the base of tissue culture plates with suspended polymer scaffold meshes (124 polymer versus POMac versus no-scaffold control) showed no visual difference in cell viability at days 2, 4, and 6 and regardless of the scaffold material. FIG. 6B. LDH (lactate dehydrogenase) cytotoxicity assay assessment of rat cardiac fibroblast cells seeded on 124 polymer films and in polystyrene wells as a control. The assay quantitatively measures lactate dehydrogenase (LDH), a stable cytosolic enzyme that is released upon cell lysis. The amount of color (or absorbance) formed is proportional to the number of lysed cells. The results demonstrate that the 124 polymer is not significantly cytotoxic since over time there is reduced absorbance as compared to the control.

FIGS. 7A, 7B, 7D, 7E, and 7F illustrate the photomicrographs of rat tissue explants which surrounded subcutaneous polymer discs of 124 polymer (left) and PLLA (right) 60 days post-implantation. Images were stained for specific markers. All stains were quantified as percentage of total area with a positive stain (*p<0.05, **p<0.1, P=polymer disc) (A) Masson's Trichrome staining presents less collagen deposition along the 124 polymer disc boundary in comparison to PLLA control. (B) Staining for all macrophages (CD68) and (C) M2 macrophages shows increased total macrophage presence in the 124 polymer compared to the PLLA group. (D) T-cell recruitment (CD3) is also heightened along 124 polymer sample boundaries compared to the control but with low absolute quantities. (E-F) Staining for vascular markers that signify vascularization, CD31 and smooth muscle actin (SMA), do not present any appreciable differences. Scale bars, 20 μm.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

All publications, patent applications, patents, figures and other references cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

### Definitions

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

An “agonist” is a drug, agent, or compound that binds to and activates its cognate receptor in some fashion, which directly or indirectly brings about a physiological effect.

An “antagonist” is an agent that binds to a receptor, and which in turn prevents binding by other molecules/

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers indicate like elements throughout. As used herein the term “and/or” includes any and all combinations of one or more of the associated listed items.

In the pharmaceutical arts, the term “efficacy” can describe the strength of a response in a tissue produced from a single drug-receptor complex. In the context of this disclosure, “efficacy” can also be defined as a response elicited by a drug or test agent that improves the phenotype of a cell or tissue.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments. Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

As used herein, the term “hydrogel” refers to a physically or chemically cross-linked polymer network that is able to absorb large amounts of water and is a common material for forming tissue engineering scaffolds. They can be classified into different categories depending on various parameters including the preparation method, the charge, and the mechanical and structural characteristics. Reference can be made to S. Van Vlierberghe et al., “Biopolymer-Based Hydrogels As Scaffolds for Tissue Engineering Applications: A Review,” Biomacromolecules, 2011, 12(5), pp. 1387-1408, which is incorporated herein by reference.

As used herein, the term “microfabrication” is a concept that includes fabrication on a nanometer or micrometer level, including microfabrication and nanofabrication. Methods for microfabrication are well known in the art. Reference to certain microfabrication techniques that may be applicable in the invention include, for example, U.S. Pat. No. 8,715,436, 8,609,013, 8,445,324, 8,236,480, 8,003,300, as well as Introduction to Microfabrication (2004) by S. Franssila. ISBN 0-470-85106-6, each of which are incorporated herein by reference.

The term “microfabricated structure” as used herein is a concept that includes one or more structures occupying a two- or three-dimensional space, including a structure fabricated on a nanometer or micrometer scale. The term “two-dimensional” means on a surface in either vertical or horizontal space.

As used herein, the term “pharmacokinetics” refers to the actions of the body on a drug. Pharmacokinetic processes include, but are not limited to, absorption, distribution, metabolism, and elimination of drugs.

As used herein, the term “pharmacodynamics” refers to the actions of a drug on the body. Because certain classes of drugs exhibit similar effects on the body, pharmacodynamic properties determine the group in which a drug or agent is classified.

As used herein, the term “POMaC” refers to poly(octamethylene maleate (anhydride) citrate) (POMaC) or the POMaC prepolymer which comprises a mixture of 1,8-octanediol, citric acid, and maleic anhydride. Reference can be made to Tran et al., “Synthesis and characterization of a biodegradable elastomer featuring a dual crosslinking mechanism,” Soft Matter, Jan. 1, 2010; 6(11): 2449-2461, which is incorporated herein by reference in its entirety.

A “test agent” is any substance that is evaluated for its ability to diagnose, cure, mitigate, treat, or prevent disease in a subject, or is intended to alter the structure or function of the body of a subject. A test agent in an embodiment can be a “drug” as that term is defined under the Food Drug and Cosmetic Act, Section 321(g)(1). Test agents include, but are not limited to, chemical compounds, biologic agents, proteins, peptides, antibodies, nucleic acids, lipids, polysaccharides, supplements, diagnostic agents and immune modulators and may also be referred to as “pharmacologic agents.”

As used herein, the term “toxicity” is defined as any unwanted effect on human cells or tissue caused by a test agent, or test agent used in combination with other pharmaceuticals, including unwanted or overly exaggerated pharmacological effects. An analogous term used in this context is.

As used herein, the term “tissue strand” refers to a three-dimensional tissue culture which is formed by first seeding a growth chamber or the like in various embodiments, e.g., the Biowire, Biotube, Biorod, or Angiotube embodiments, wherein the growth chamber comprises one or more suspended growth surfaces, e.g., wires, tubes, sufficient for growing a tissue strand. The tissue strand can grow or form around a single growth surface, e.g., a polymer wire or tube, or the tissue strand can grow between one or more growth elements, e.g., as in the Biorod embodiment.

As used herein, the term “tuneability” as it is used in reference to a “tunable” polymer, e.g., 124 polymer, refers to the capability of adjusting the process of polymerization of a polymer in a manner that allows for the formation of a resultant polymer product to have different mechanical and/or physical properties, such as elasticity, stiffness, and/or reactivity, or other properties. This concept is referred to in the context of certain polymers, such as the 124 polymer, that may be advantageously used in various embodiments/devices of the present invention to form the various components of the devices of the invention, e.g., polymer wires, scaffolds, scaffold layers, and other components. Tuneable polymers, such as the 124 polymer, may have adjustable or “tuneable” properties by adjusting, for example, (a) the degree, quantity, intensity, or timing of UV crosslinking to affect the numbers of intermolecular bonds that link together a plurality of units of the polymers described herein (i.e., the elastomer monomer units such as the 124 polymer), (b) the ratio of pre-polymer units that form the polymer, e.g., the ratios of 1,8-octanediol, citric acid, and maleic anhydride in the case of POMaC or the ratios of 1,8-octanediol, 1,2,4-butanetricrboxylic acid and the maleic anhydride in the case of the 124 polymer, (c) the degree of intermolecular polymerization via intermolecular polycondensation reactions between neighboring acidic groups on the 1,2,4-butanetricarboxylic acid groups, and (d) the extent and/or size of pore formation in the resulting elastomer, which are formed by the use of porogens (e.g., polyethylene glycol dimethyle ether (PEGDME)) during elastomer formation. Each of these parameters are adjustable and constitute the tuneability aspect of the elastomer's final physical characteristics.

### Dual-Crosslinkable Pre-Polymer

In one aspect, the present invention relates to a novel polymer (or pre-polymer) that may be used to construct an elastomer via dual-crosslinking functionalities for use in tissue engineering as a material for the construction of tissue scaffolding components. In particular embodiments, the polymer is especially suited for forming elastomers that replicate the elastomeric properties of native cardiac tissue; and thus, which are particular suited for use in cardiac tissue engineering. A network of polymers may be formed, i.e., an elastomer, through a dual-crosslinking mechanism that includes UV crosslinkable moieties, as well as intermolecular ester-bond formation between neighboring acid groups. The extent of dual-crosslinking may be controlled through various parameters (e.g., conditions controlling UV crosslinking and conditions controlling intermolecular ester-bond formation) to “fine-tune” the resulting properties of the elastomer (including mechanical properties, biodegradability, and functionality).

Thus, in one aspect, the invention describes a new biodegradable polymer capable of forming an elastomer through a dual-crosslinking mechanism and methods of making and using this polymer and the resulting elastomer for tissue engineering, and in certain embodiments, cardiac tissue engineering.

As used herein, the term “polymer” refers to the building blocks used to construct the ultimate elastomers of the invention, which are networks of interconnected individual polymers that have been joined via a dual-crosslinking mechanism that includes UV crosslinks and crosslinks formed by intermolecular condensation reactions. The term “pre-polymer” is intended as an equivalent term to “polymer” and is in reference to the polymer or pre-polymer as a monomer unit of the ultimate elastomer of the invention, which can be viewed also as a polymer of individual pre-polymer subunits joined through UV crosslinks and ester-bond formations.

In certain embodiments, the polymer or pre-polymer is poly(octamethylene maleate (anhydride) 1,2,4-butanetricarboxylate) and which is capable of undergoing intermolecular crosslinking to form an elastomer, i.e., a plurality of polymer units linked together to form a network of molecules having elastomeric properties. The crosslinking can be formed by UV crosslinking or intermolecular polycondensations reactions or some combination thereof.

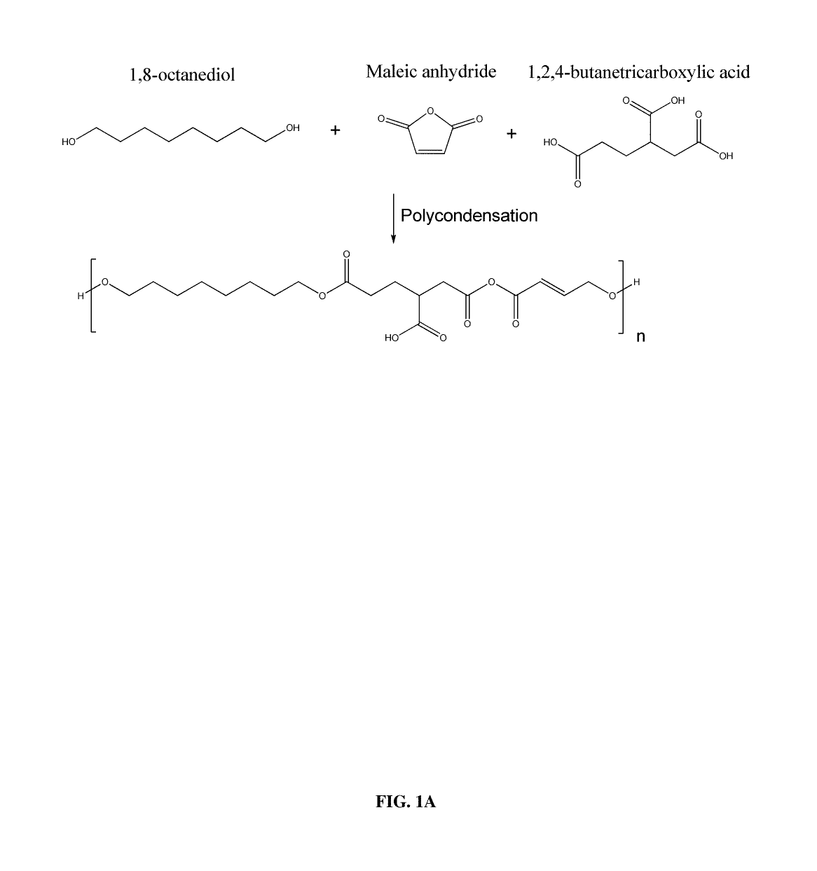

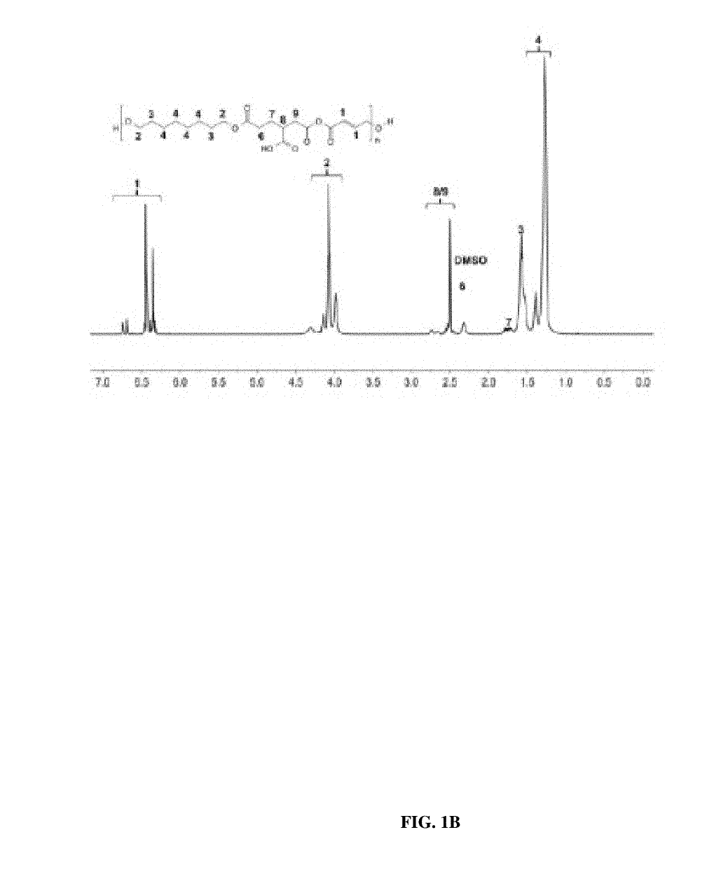

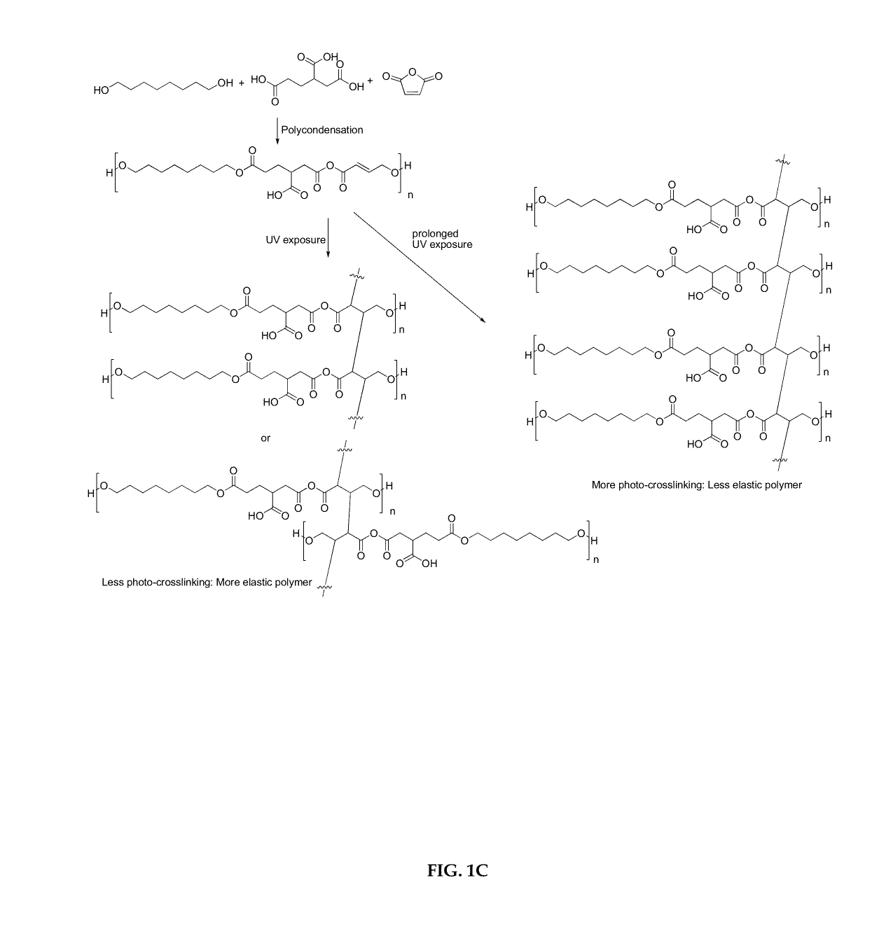

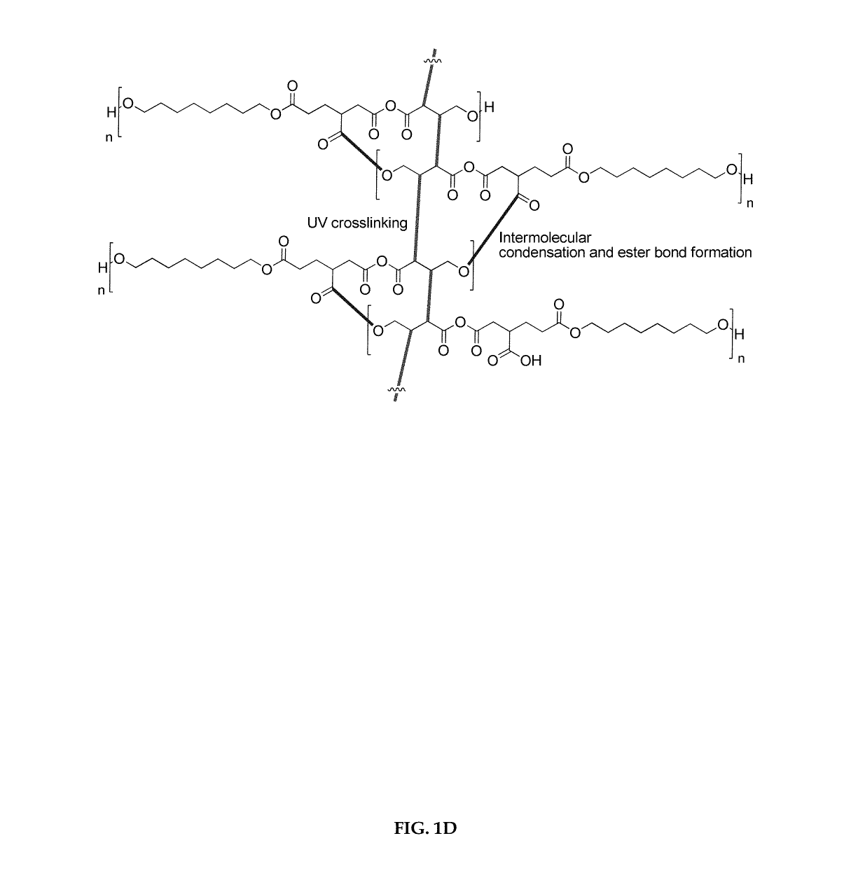

In various embodiments, the crosslinking is formed as a result of exposing the initial polymers (i.e., a solution of polymers) with ultraviolet (UV) light at a suitable intensity and for a suitable period of time sufficient to form a desired number of photocrosslinks. In certain embodiments, the photocrosslinks are covalent bonds formed between a pair of carbon atoms—one each from two different polymers—that correspond to the double-bonded carbons of the maleic acid component. That is, under UV radiation, the double carbon bonds in the maleic acid component of two separate and proximal polymers of the invention undergo a reaction whereby each of the double bonds are converted to a single carbon-carbon bond, with a new intermolecular carbon-carbon bond forming between two maleic acid components. In one embodiment, UV crosslinking is illustrated in FIG. 1C. As shown in the FIG. 1C, the extent of UV crosslinking (i.e., fine-tuning) can be adjusted by varying the various parameters, including the intensity of the UV radiation, and/or the timing of exposure.

In various other embodiments, the crosslinking may also form as a result of intermolecular polycondensation as between two tricarboxylic acid components of two separate but adjacent polymers. With this type of crosslinking, a condensation reaction (i.e., the removal of water) forms an ester bond is formed between two neighboring acid components of the tricarboxylic acid moieties of two different polymers, with the concomitant removal of water. This reaction and bond formation is depicted in FIG. 1D. The extent of intermolecular polycondensation can be controlled by various factors, such as, temperature, pressure (vacuum conditions), the presence of chemical reactivity protective groups, etc. For example, in the synthesis of 124 polymer, intermolecular polycondensation would be more effective between 140-170° C. for the production of the viscous pre-polymer. Polycondensation will be limited at temperatures <50° C. and completely blocked at temperature less than 0° C.

Both types of crosslinking may operate together as a dual-crosslinking system to form elastomers of the invention having a range of mechanical properties (e.g., stiffness, flexibility) and other characteristics (e.g., biodegradability).

In a particular embodiment, the invention relates to a biodegradable polymer made by the following polycondensation reaction scheme involving the following three reactants or their equivalents:

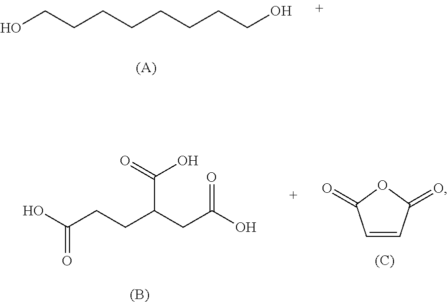

Wherein; reactant A is at least one diol, e.g., 1,8-octanediol or its equivalents; reactant B is at least one tricarboxylic acid, e.g., 1,2,4-butanetricarboxylic acid or its equivalents; and reactant C is at least one unsaturated di-acid, e.g., maleic anhydride or its equivalents, wherein the resulting polymer is represented by Formula I (poly(octamethylene maleate (anhydride) 1,2,4-butanetricarboxylate)):

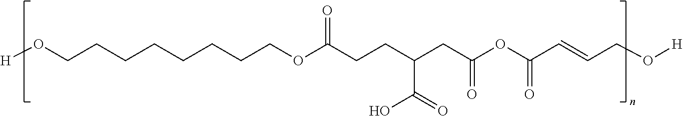

or an equivalent thereof.

Thus, the present invention describes, a new family of biodegradable polymers represented by the exemplary Formula I which have been created with many advantages over the current materials, such as POMaC. As compared in particular to POMaC, the advantages of the elastomers of the present invention include:
a. highly elastic properties under aqueous conditions;
b. tunable elastic properties of resulting elastomer according to UV light exposure, monomer composition, and porosity;
c. dual-crosslinkability, including both UV crosslinking (e.g., UV light-induced radical polymerization) and polycondensation reaction between neighboring acidic groups (one of the major advantages of the polymers of the invention is that after UV crosslinking, the polymer remains saturated with functional groups (—COOH, —OH), which can be used for further ester-bond crosslinking via polycondensation or bioconjugation);
d. elastomeric properties easily tunable within the range of adult heart myocardium (e.g., compatible Young's modulus);
e. stable degradation characteristics (including both hydrolytically and in a cellular environment); and
f. good cell attachment characteristics (e.g., cardiac cell attachment), among other advantages which make the elastomeric material of the invention highly suitable for use as a scaffold material in engineered tissue constructs, and in particular, in engineered cardiac tissue engineered constructs.

In various embodiments, the present invention provides a dual crosslinkable biodegradable polymer composition having a multifunctional monomer; a diol; and an unsaturated di-acid at least partially polymerized to form a network and photocrosslinked into a dual crosslinked polymer network. The multifunctional monomer includes a tricarboxylic acid, a diol, and the unsaturated diacid (e.g., maleic anhydride). The network may be crosslinked by polycondensation or free radical polymerization and one or more crosslinkers may be optionally added.

In another embodiment, the present invention utilizes inexpensive and non-toxic monomers for synthesis of elastomer, such as reactants A, B, and C above, or equivalents thereof.

In one embodiment, the invention provides that a crosslinked elastomer pre-polymer is a polyester formed from monomers of tricarboxylic acid, at least one diol, and at least one unsaturated di-acid through polycondensation reaction.

In another embodiment, the invention provides an elastomer formed by UV-cross-linking a pre-polymer or polyester resulting from a polycondensation reaction of monomers of 1,2,4-butanetricarboxylic acid, at least one diol (e.g., 1,8-octanediol) and at least one unsaturated di-acid (e.g., maleic anhydride). The network of pre-polymers comprising UV crosslinks may be further joined by intermolecular ester-bond formation between neighboring tricarboxylic acid groups by intermolecular polycondensation reactions.

In order to meet the versatile needs for suitable biomaterials, in particular when it comes to cardiac tissue engineering applications, the choice of monomer for polymer synthesis is very important since it may affect the biocompatibility, biodegradability, and the cell affinity of polymers to be synthesized. Monomers such as citric acid [20-22], fumaric acid [1] maleic acid [23-26], maleic anhydride [27], 1,8-octanediol [21, 22], and PEG [11, 28, 29], have been extensively used in the field of biomaterials. The monomers used in the current invention disclosure for synthesizing new biomaterials include: 1) tricarboxylic acid (e.g., 1,2,4-butanetricarboxylic acid), which can form crosslinking networks with other difunctional monomers; 2) various unsaturated diacids or anhydrides such as fumaric acid, maleic acid, maleic anhydride, including especially maleic acid; 3) various diols including saturated aliphatic diols such as C2-C12 diols and macrodiols such as poly(ethylene glycol) (PEG), including especially 1,8-octanediol.

FIG. 1A is a representative schematic of the synthesis of an exemplary pre-polymer of the invention, namely, (poly(octamethylene maleate (anhydride) 1,2,4-butanetricarboxylate)) of Formula I. As depicted, the tricarboxylic acid, diol, and unsaturated di-acid are reacted to form a prepolymer via condensation. The prepolymer can be further crosslinked through photocrosslinking and/or polycondensation. (See FIGS. 1C and 1D).

Without being limited by theory, one rationale behind the biomaterial design of the invention include: 1) Finding clues from nature, the components of the extracellular matrix (ECM) such as collagen and elastin are crosslinked polymers and elastic within certain deformation. Thus, we chose crosslinking as a mechanism to confer elasticity to the biomaterials; 2) Due to the advantages of using inexpensive monomers and a cost-effective synthesis, we chose polycondensation for pre-polymer synthesis simply under heating without using any catalyst. Tricarboxylic acid, maleic acid, various aliphatic diols, and macrodiols (Polyethylene oxide) are all inexpensive, commercially available (e.g., SIGMA®), and have been used in many other biomaterial syntheses for tissue engineering uses. 3) The feeding ratio of the monomers, choices and combination of aliphatic and macro-diols, and polymerization degree can be varied to tune the mechanical and degradation properties and functionality of the resulting polymers. 4) The dual-crosslinking mechanism allows for tuneability of the resulting elastomer. Maleic acid introduces photocrosslinkable double bonds in the prepolymers. The tricarboxylic acid moiety is a multifunctional monomer with side —COOH and —OH. Photocrosslinking provides strong C—C crosslinking bond in the polymer network leaving the polymer rich in unreacted —COOH and —OH groups for further bioconjugation or polycondensation crosslinking through degradable ester bonds formed by —COOH and —OH.

In various embodiments, the material properties such as swelling, thermal behavior, mechanical properties, and degradation rate are dependent on polymer compositions, molecular weights, crosslinking degree etc. It has been shown that for photocrosslinking reaction, the choice and concentration of the photoinitiator, the light intensity, and the amount and locations of reactive double bonds in the formulation also affect the rate of polymerization. In this disclosure, we will demonstrate the potentials of 124 polymer and resulting elastomers (upon crosslinking) on their uses in tissue engineering, drug delivery and wound dressing applications. This disclosure describes a new type of biodegradable crosslinked polyester network family, which can be used as platform biomaterials for a variety of bioengineering applications.

### Polycondensation Reaction

The reaction conditions for the initial polycondensation reaction involving the simultaneous condensation of the diol (e.g., 1,8-octanediol), tricarboxylic acid (e.g., 1,2,4-butanetricarboxylic acid), and unsaturated di-acid (e.g., maleic anhydride) may be adjusted. In particular, reaction conditions including temperature, solvent choice, stirring conditions, and reactant ratios, as well as any other typical reaction parameters (e.g., pH, buffer, salt concentration), may be changed.

The reaction may be conducted at any suitable temperature. The temperature may be fixed or may be varied during the course of the reaction. Suitable temperatures include between 125-135° C., or between about 130-145° C., or between about 140-155° C. , or between about 145-165° C. , or between about 150-175° C., or higher. In a preferred embodiment, the temperature is set at about 150° C.

The ratio of the monomers may also be adjusted.

In one embodiment, the ratio of the diol to the tricarboxylic acid can be about 0.01 to 1, about 0.05 to 1, about 0.10 to 1, about 0.20 to 1, about 0.30 to 1, about 0.50 to 1, about 0.60 to 1, about 0.70 to 1, about 0.80 to 1, about 0.90 to 1, about 1:1, 1:2, or about 1:3, or about 1:4, or about 1:5, or about 1:6, or about 1:7, or about 1:8, or about 1:9, or about 1:10, or up to about 1:25, or 1:50, or 1:100, or more.

In another embodiment, the ratio of the diol to the unsaturated di-acid can be about 0.01 to 1, about 0.05 to 1, about 0.10 to 1, about 0.20 to 1, about 0.30 to 1, about 0.50 to 1, about 0.60 to 1, about 0.70 to 1, about 0.80 to 1, about 0.90 to 1, about 1:1, 1:2, or about 1:3, or about 1:4, or about 1:5, or about 1:6, or about 1:7, or about 1:8, or about 1:9, or about 1:10, or up to about 1:25, or 1:50, or 1:100, or more.

In still another embodiment, the ratio of the tricarboxylic acid to the unsaturated di-acid can be about 0.01 to 1, about 0.05 to 1, about 0.10 to 1, about 0.20 to 1, about 0.30 to 1, about 0.50 to 1, about 0.60 to 1, about 0.70 to 1, about 0.80 to 1, about 0.90 to 1, about 1:1, 1:2, or about 1:3, or about 1:4, or about 1:5, or about 1:6, or about 1:7, or about 1:8, or about 1:9, or about 1:10, or up to about 1:25, or 1:50, or 1:100, or more.

The timing of the reaction may also be adjusted and may be anywhere from about a minute to several minutes, or for about 5-15 min, or about 10-25 min, or about 20-35 min, or about 30-45 min, or about 40-55 min, or about an hour, or more than hour, such as 1-2, or 2-3, or more hours.

The resulting pre-polymer can be in the form of a viscous solution. The pre-polymer may be freeze-dried. In other embodiments, the pre-polymer viscous solution may be injected into a mold prior to forming the elastomer (i.e., prior to cros slinking) in order to form a specified and desired tissue scaffold shape, e.g., the formation of a tube, a filament, a sheet, or a three-dimensional structure of any variety of shapes and sizes as needed which can depend on the particular tissue engineering application of interest.

### Elastomer Formation (Dual Crosslinking)

The final pre-polymer may be converted to an elastomer (e.g., the “124 polymer”) by UV crosslinking. In certain embodiments, further crosslinks may be introduced by ester-bond formation between neighboring tricarboxylic acid groups via a further condensation reaction.

### Photocrosslinking

Any suitable method for photocrosslinking can be used. As intended herein, photocrosslinking refers to the photoinduced formation of a covalent bond between two macromolecules or between two different parts of one macromolecule. In various embodiments, the photocrosslinking introduces a covalent bond between the double bond carbons of the unsaturated di-acid group, with a concomitant loss of the double bond in favor of the formation of an intermolecular carbon-carbon bond.

In one embodiment, the pre-polymer may be crosslinked by free radical UV-light induced polymerization. The purified pre-polymer can be dissolved in a solvent, such as, dimethylsulfoxide (DMSO). To generate free radicals, a photo initiator such as but not limited to (PI) 2-hydroxy-1-[4(hydroxyethoxy)phenyl]-2-methyl-1 propanone (Irgacure 2959) can be dissolved in the polymer solvent solution. It will be appreciated that other photoinitiators are well known in the art. A photoinitiator is a compound especially added to a formulation to convert absorbed light energy, UV or visible light, into chemical energy in the form of initiating species, free radicals or cations. Based on the mechanism by which initiating radicals are formed, photoinitiators are generally divided into two classes: Type I photoinitiators undergo a unimolecular bond cleavage upon irradiation to yield free radicals; Type II photoinitiators undergo a bimolecular reaction where the excited state of the photoinitiator interacts with a second molecule (a coinitiator) to generate free radicals. UV photoinitiators of both Type I and Type II are available. However, visible light photoinitiators belong almost exclusively to the Type II class of photoinitiators. Photoinitiators are available commercially from sources such as SIGMA®.

The solution optionally including the photoinitiator can be poured into a Teflon mold and placed under a UV light source, e.g., UVP 365 nm Long Wave Ultraviolet Lamp (Upland, Calif.) for a suitable period of time, e.g., about 3 minutes. Any suitable UV light source or lamp may be used and such systems are well known and readily available in the art.

The time and intensity of the UV exposure may be adjusted as needed. In general, the number of photocrosslinks will increase with any of: (a) increasing UV exposure time; (b) increasing the intensity of the UV light; or (c) a combination of increased time and light intensity. An increased number of photocrosslinks generally corresponds with increased stiffness and reduced flexibility. The number of photocrosslinks can also impact the biodegradability of the resulting elastomer.

Prior to UV crosslinking, the pre-polymer material can be introduced into a mold to provide a specific two-dimensional or three-dimensional shape that is suitable for the intended tissue engineering application.

In addition, the pre-polymer material can be introduced, e.g., by injection, directly into the body at a desired site (e.g., an site of injury in need of tissue repair) and then subsequently converted from the pre-polymer form to a crosslinked elastomer under in situ conditions.

### Ester-Bond Intermolecule Crosslinking (“Secondary” Polycondensations)

One of the advantages to this new family of polymers is that they offer a dual crosslinking mechanism. Prior to or following the photocrosslinking, the polymer still contains many unreacted hydroxyl and carboxylic groups available for further post-polymerization via ester-bond formation, which allows for continued control over the mechanical properties of the polymer. The photocrosslinked polymer can be heated at a temperature range from 60-120° C. for times ranging from minutes to hours even week with or without vacuum in an oven to further crosslink the network. All the pre-polymers can be crosslinked directly via ester-bond cros slinking even without a pre-photocrosslinking. Thus, this dual-crosslinking mechanism offers a great flexibility to generate a family of polymers with wide controls on material properties.

### Polymer/Elastomer Characterizations

Any suitable methods can be used to evaluateand/or determine the mechanical, chemical, or other functional characteristics of the polymers and elastomers of the invention, and accordingly, to facilitate the optimal fine-tuning conditions that produce a desired elastomer. Analytical methods include but are not limited to the following:

Fourier Transform Infra-Red Spectroscopy (FT-IR): To analyze the functional groups present in the pre-polymer, FT-IR spectroscopy measurements can be recorded at room temperature using a Nicolet 6700 FT-IR (Thermo Scientific, Waltham, Mass.) (or other suitable device) for the 124 polymer. Pre-polymer samples can be prepared by a solution-casting technique. Briefly, a 5% pre-polymer solution in 1,4-dioxane can be placed on a potassium bromide crystal and dried overnight in a vacuum hood. Other equivalent methods are envisioned.

Proton Nuclear Magnetic Resonance (1H-NMR): A 1H NMR (250 MHz JNM ECS 300, JEOL, Tokyo, Japan) can be used to analyze the structure of the polymers. In one embodiment, The pre-polymers were purified twice as mentioned above and then dissolved in dimethyl sulfoxide-d6 (DMSO-d6) to make a 3% pre-polymer solution and placed inside a 5-mm-outsidediameter tube. The chemical shifts for the 1H-NMR spectra were recorded in partsper-million (ppm), and were referenced relative to tetramethylsilane (TMS, 0.00 ppm) as the internal reference. Other equivalent methods are envisioned.

Differential Scanning calorimetry (DSC) and Thermogravimetric Analysis (TGA): In one embodiment, the thermal behavior of the polymer of different ratios has been studied on a DSC550 (Instrument Specialists Inc., Spring Grove, Ill.) and TGA (Thermogravimetric Analysis, Mettler Toledo, Columbus, Ohio). For the DSC analysis, samples are first scanned up to 150° C. with a heating rate of 10° C./min under nitrogen purge (50 ml/min) to remove any trace water from the sample. Thereafter, the sample was cooled with a cooling rate of −40° C./min to −60° C. The sample was then scanned a second time up to 230° C. The glass transition temperature (Tg) is determined as the middle of the recorded step change in heat capacity from the second heating run. TGA thermograms have been observed under the flow of nitrogen gas (50 ml/min) at a scanning speed of 101° C./min in the range of 50-600° C. The decomposition temperature (Td) was defined as the temperature at which 10% weight loss of the samples occurred. Other equivalent methods are contemplated.

Tensile Mechanical Properties: In one embodiment, tensile mechanical properties have been studied according to the ASTM D412a standard on an MTS Insight II mechanical tester equipped with a 500 N load cell (MTS, Eden Prairie, Minn.). Crosslinked 124 polymer films can be cut using a dog-boneshaped (26 mm×4 mm×1.5 mm; length×width×thickness) aluminum die. The dog-bone-shaped films were then subjected to the stress of 500 N at a rate of 500 mm/min. Obtained values were converted to a stress-strain curve and the initial slope (0-20% of the curve) has been used to calculate the Young's modulus. A detailed study of the effect of different ratios of the monomers, the amount of PI, and concentration of the polymer in solvent on mechanical properties of the polymer were studied. Other equivalent methods are contemplated.

Young's Modulus for Mechanical characterization: In one embodiment, a three-factor design of experiment (DOE) was conducted to evaluate the range of modulus that 124 polymer can achieve as a result of variations in the preparation procedure. The three factors considered were: (A) monomer feed ratio of 1,2,4-butanetricarboxylic acid and maleic anhydride (124:MA); (B) UV exposure energy dose (mJ) and (C) porous fraction, achieved by the incorporation of porogen into the pre-polymer which is leached out after curing. Batches of pre-polymer were prepared according to the design matrix. A Pre-polymer was injected into a polydimethylsiloxane mold before the curing step, where each sample was then individually exposed to the experiment-prescribed UV exposure energy dose. Samples were designed as thin strips (length: 10 mm, width: 1.5 mm, thickness: 0.1 mm) according to an ASTM D882 standard modified to be compatible with the equipment available in the laboratory for conducting tensile tests. Samples were collected and soaked in PBS for a minimum of 24 hours before tensile testing. Tensile tests were conducted in PBS with a Myograph (KENT SCIENTIFIC®). Elasticity was taken as the slope of the linear portion of the generated stress-strain curve and the test was carried out to failure.

Transwell assay for polymer degradation: In one embodiment, the polymer degradation was measured by exposing pre-polymer strips (124, POMaC, 124+40% porogen, POMaC+40% porogen) (1.5 mm×0.5 mm×10 mm) to UV (365 nm) energy. The strips were weighed in sets of 8 to determine the initial mass. Strips were soaked in PBS for 2 hours, 70% ethanol overnight, and washed twice with sterile PBS. These were then placed in base of transwell insert 24-well plates (Corning), with rat-CM seeded in the transwell inserts and cultivated in rat CM media. The strips were collected at days 1 and 14, washed twice in deionized distilled water and dried under lyophilization for two days. Final mass was measured, recorded, and expressed as a percentage loss of the initial dry mass (day 0). Strips were also characterization for a change in mechanical properties, as described in mechanical characterization.

Hydrolytic degradation: In one embodiment, UV cured polymer strips, as described in transwell degradation, were placed in pre-weighed 20 mL glass scintillation vials in sets of 8 and the initial dry mass was recorded. 10 mL of PBS was added to each vial, which was sealed and placed in 37° C. environment under agitation. Vials were collected at days 1, 7, and 30, dried under lyophilization for 2 days and the final dry mass was recorded. Degradation was reported as percentage of initial mass lost.

Swelling Properties: In one embodiment, 124 polymer hydrogels can be fabricated according to the process mentioned above. In order to investigate the effect of the amount of PI on the swelling properties, different concentrations of Irgacure 2959 (1%, 2%, 3%, 4% and 5% by weight) have been used to photocrosslink the polymer. In order to observe the effect of polymer concentrations in solvent, different polymer concentrations (20%, 50% and 100% by weight) has been used in the study. DMSO can be used as the swelling agent in this study due to its high boiling point, and deionized water was used to observe the water uptake of the polymer. Different ratios of the polymer can be used to study the swelling in phosphate buffered solution (PBS pH 7.4) at 37° C. with 2% of PI and 50% concentration of polymer in solvent. Six polymer discs (7 mm in diameter) were cut from the crosslinked films using a cork borer. The discs were allowed to swell in DMSO until the equilibrium state has been achieved (48 hours). The surface of the swollen discs was gently blotted with filter paper to remove any excess swelling agent, and the sample was then weighed (MW). The discs were then placed in water to exchange the DMSO for twenty-four hours, and freeze-dried for seventy-two hours. The samples were then weighed to determine the dry weight (Md). Equilibrium swelling ratio was calculated as (Mw−Md)/Md. The experiment was repeated three times and the average value was reported. Similarly, water uptake and swelling in PBS has been done using the same procedure. Other equivalent methods are contemplated.

Sol Gel Fraction: In one embodiment, six polymer discs (7 mm in diameter) were cut from the crosslinked films using a cork borer. The discs were weighed to find the initial mass (Mi), and suspended in DMSO for twenty-four hours. The DMSO was changed every six hours. Next, the gels were placed in water to exchange DMSO for twenty-four hours and freeze-dried for seventy-two hours. The dried samples, absent of unreacted polymer, were weighed to obtain (Md). The sol gel fraction was calculated as (Md−Mi/Mi)*100. The experiment was repeated three times and average value was reported. Other equivalent methods are contemplated.

Degradation: In one embodiment, six polymer discs (7 mm in diameter) were cut from crosslinked films using a cork borer. The discs were weighed to find the initial mass (Mi), and suspended in PBS (pH 7.4) and kept at 37° C. The pH was monitored regularly to maintain a constant pH. At the desired time point, the samples were rinsed with deionized water, freeze-dried, and weighed to find the remaining mass (Mt). Percent mass loss was calculated as [(Mi−MO/Mi]*100. In addition to PBS, an accelerated degradation study using 0.05M NaOH was also used to degrade the polymer films. Other equivalent methods are contemplated.

Biocompatibility of 124 polymer in vitro cell culture: In one embodiment, six polymer discs (7 mm in diameter) were cut from crosslinked films using a cork borer. The discs were soaked in DMSO to remove the sol fraction, soaked in water to remove the DMSO, and freeze-dried. The discs were then sterilized in 100% ethanol for 30 minutes, and placed under UV light for another 30 minutes. The disks were seeded with 5,000 cells/cm2 (NIH 3T3 Fibroblasts). The cells were allowed to proliferate in DMEM culture media with 10% fetal bovine serum (FBS) for 3 days. The cells were then fixed in gluteraldehyde and H&E stained. Other samples were fixed in gluteraldehyde, freeze-dried, gold sputter coated, and viewed under a Hitachi S-3000N SEM (Hitachi, Pleasanton, Calif.). Other equivalent methods are contemplated.

Foreign body response: In one embodiment, 124 polymer disks (7 mm in diameter, 2 mm of thickness) were implanted in 7-week-old female Sprague-Dawley rats by blunt dissection under deep isoflurane-O2 general anesthesia. Animals were cared for in compliance with the regulations of the animal care and use committee of The University of Texas at Arlington. POMC samples were implanted symmetrically on the upper and lower back of the same animal. The rats were sacrificed and tissue samples surrounding the implants were harvested with intact implant at 1 week and 4 weeks. The samples were sectioned and stained with hematoxylin and eosin (H&E). Other equivalent methods are contemplated.

Any additional standard methodologies may be used to assess, measure, and evaluate the mechanical properties, chemical functionality, elasticity, biodegradability, stability, and any other physical and/or mechanical and/or chemical properties of the pre-polymer (e.g., 124 polymer) or elastomers thereof (formed by photocrosslinking and/or intermolecular ester-bond formation of the initial pre-polymer). The above methodologies and specific embodiments are not intended to be limiting in this regard.

### Tissue Culture Systems and Scaffolds

The 124 pre-polymer and resulting dual-crosslinked elastomers can be used in connection with making a range of tissue culture devices, in particular, cardiac tissue culture devices.

The present invention contemplates various tissue culture systems for making and using three-dimensional biological tissues that accurately mimic native physiology, tissue architecture, vasculature, and other properties of native tissues. The mimicked tissues may include, but are not limited to, cardiac, hepatic, neural, vascular, kidney, and muscle tissues. The methods, composition, and devices may be used in a variety of applications that include drug testing, tissue repair and/or treatment, and regenerative medicine. The tissue culture devices of the invention can be used particularly for methods that include: (a) the testing of the efficacy and safety (including toxicity) of experimental pharmacologic agents (including, but not limited to, small molecule drugs, biologics, nucleic acid-based agents), (b) the defining of pharmacokinetics and/or pharmacodynamics of pharmacologic agents (including, but not limited to, small molecule drugs, biologics, nucleic acid-based agents), (c) characterizing the properties and therapeutic effects of pharmacologic agents (including, but not limited to, small molecule drugs, biologics, nucleic acid-based agents) on a subject, (d) screening of new pharmacologic agents, (e) provide implantable engineered tissues for use in regenerative medicine for treating damaged and/or diseased tissues, (e.g., use of the tissue constructs, devices, and/or systems of the disclosure to study cardiac disease states, including patients with electrical conduction defects (iPSC-CM)), and (f) personalized medicine.

In one aspect, the present disclosure provides a bioreactor system that may combine architectural and electrical cues to generate a microenvironment conducive to maturation of three-dimensional (3D) cardiac tissues or other contractile tissues. The present disclosure also provides methods and techniques for fabricating the disclosed devices, for using the disclosed devices to cultivate tissues, and for using the resulting tissues for implantation and other applications.

Another aspect relates to a bioreactor system in which cells are seeded in a hydrogel, e.g., a collagen gel, around a scaffold (e.g., a template suture) in a bioreactor channel or chamber (e.g., a microfabricated well). The seed cells in this example (in particular, where the cells have electrical characteristics, such as cardiac cells) can be subjected to electrical field stimulation according to a defined regimen defining specific frequency of stimulation at specific times (e.g., progressive frequency increase over several days). Consistent with maturation, the generated tissues (e.g., cardiac tissues) exhibit a significant degree of ultrastructural organization, improved conduction velocity and enhanced Ca2+ handling and electrophysiological properties.

In other aspect, perfusable bioreactor systems may be generated in which a perfusable scaffold having a lumen (e.g., a tubing template, such as a polytetrafluoroethylene (PTFE) tubing template) is suspended in the bioreactor channel or chamber (e.g., a microfabricated bioreactor channel). The scaffold may provide guidance for cells to align and elongate. To demonstrate the feasibility of such a device for drug testing, nitric oxide (NO) can be supplied in the cell culture channel to provide biochemical stimulation to cardiomyocytes within the cardiac tissue. NO was released from perfused sodium nitroprusside (SNP) solution and perfused from the scaffold lumen to the tissue culture (e.g., NO passed through the tubing wall) to reach the tissue space with cardiomyocytes. An example of the disclosed bioreactor device can also be integrated with electrical stimulation, which may further improve phenotype of the cells, e.g., cardiomyocytes.

In other aspects, the disclosure provides devices that enable measurement of the contraction force of cultivated tissues. In some examples, the device may have a multi-well configuration (e.g., configured as a 96 well plate), which may enable the device to be compatible with drug screening and/or non-invasive on-line monitoring of function. The 124 pre-polymer and resulting dual-crosslinked elastomers are particularly suited for this use as the mechanical-physical properties of the 124 material is similar to adult cardiomyocytes.

In still other aspect, the disclosure provides a hybrid approach to create a microfluidic tissue. Such an approach may include providing an example device having a 3-D branched micro-channel network with thin channel walls to provide mechanical support to the built-in vasculatures (e.g., composed of the biodegradable elastomer, (124 polymer). A hydrogel (e.g., collagen based hydrogel) embedded with seed cells (e.g., cardiac cells) may be cast around the network such that the cardiac cells may remodel the aqueous matrix and compact around the built-in vasculature of the 3-D network to form macroscopically contracting vascularized cardiac muscle with physiological cell density. The resulting branched tissue, or the branching permeable polymer scaffold alone, may be used for implantation.

In still other aspects, the disclosed bioreactor systems may be similar to or reproduce the complexity of the native tissue architecture in vitro, thus enabling the cultivated cells to assume the structure which they would be expected to assume in vivo. Reproducing this structure may enable the cells to mature and to assume a similar function they would have in vivo. In various examples, the disclosed devices may be suitable for culture of muscle cells such as cardiomyocytes, skeletal muscle cells, smooth muscle cells as well as excitable tissues such as neurons and cells that may require rich vasculature such as hepatocytes, among others. In various examples, the disclosed devices may be suitable for drug-testing in vitro, for building a human-on-a-chip with several different compartments as well as for direct anastomosis and/or implantation into an animal or a human patient, among other applications. Implantation may include using the permeable polymer scaffold alone as a surgical cuff, bypass graft, fistula or arterio-ventricular shunt, among others. Implanting the cultivated tissue with direct anastomosis (e.g., in the form of an arterio-ventricular shunt) or without direct anastomosis at the desired target tissue location may be also possible.

In each of these cases, the 124 pre-polymer and resulting elastomer can be as a scaffold material for these devices. Further description of these devices can be found in PCT application No. PCT/CA2014/051046, which is incorporated herein by reference.

### Cells and Tissues

The 124 pre-polymer and resulting elastomer (i.e., following crosslinking of the pre-polymer to form the 124 polymer) can be used as a scaffold material for growing and forming tissue cultures based on virtually any cell, so long as the cell is capable of adhering to and growing on the scaffold.

Thus, the disclosed devices may be used in conjunction with tissues derived from any cell, such as cells from cardiac tissue, skeletal muscle tissue, smoot muscle tissue, liver tissue, kidney tissue, cartilage tissue, skin, bone marrow tissue, or combinations of such tissues, or the like. The cells used to grow the three-dimensional tissues can be sourced from anywhere, including from any commercial source, or even sourced from individual subjects or patients. For example, a tissue strand of the invention may be grown starting from a seed of a commercially available liver cell line. In another example, a tissue strand of the invention may be grown starting from a seed of cells obtained directly from a subject, e.g., cells isolated from a biopsy. In other embodiments, the three-dimensional tissues of the invention can be grown from a mixture of different cells. Such mixtures of cells can include mixtures of healthy or diseased cells from the same or different tissues, mixtures of cells from different sources or patients, or mixtures of cells from both patients and from commercial sources. The cells used to grow the tissues of the invention can also be genetically engineered cells, such as drug-resistant or drug-sensitive engineered cell lines, or other types of genetically engineered cells, including those that express various biomarkers, such as GFP.

In a particular embodiment, the three-dimensional tissues of the invention prepared by any device contemplated herein may be prepared or grown using cardiomyocytes, e.g., human cardiomyocytes. The cardiomyocytes can be obtained commercially from sources such as GE Healthcare Lifesciences, 3H Biomedical, Sciencell Research Laboratories. The cells may be characterized as expressing particular markers, such as, for example b-myosin heavy chain; a-cardiac actin; Troponin I; Troponin T; the muscle-specific intermediate filament protein, desmin; the cardiomyocyte-specific peptide hormone, atrial natriuretic peptide (ANP); and coupled gap junction proteins, connexin-43 and connexin-40.

In other embodiments, the cells used to grow the three-dimensional tissues of the invention can be stem cells, including embryonic stem cells (“ESCs”), fetal stem cells (“FSCs”), and adult (or somatic) stem cells (“SSCs”). The stem cells, in terms of potency potential, can be totipotent (a.k.a. omnipotent) (stem cells that can differentiate into embryonic and extra-embryonic cell types), pluripotent stem cells (can differentiate into nearly all cells), multipotent stem cells (can differentiate into a number of cell types), oligopotent stem cells (can differentiate into only a few cell types), or unipotent cells (can produce only one cell type). Stem cells can be obtained commercially, or obtained/isolated directly from patients, or from any other suitable source.

As used herein, a “less developmentally potent cell” is a cell that is capable of limited multi-lineage differentiation or capable of single-lineage, tissue-specific differentiation, for example, an untreated mesenchymal stem cell can differentiate into, inter alia, osteocytes and chrondrocytes, i.e., cells of mesenchymal lineage, but has only limited ability to differentiate into cells of other lineages (e.g., neural lineage.).

As used herein, a “more developmentally potent cell” is a cell that is readily capable of differentiating into a greater variety of cell types than its corresponding less developmentally potent cell. For example, a mesenchymal stem cell can readily differentiate into osteocytes and chrondrocytes but has only limited ability to differentiate into neural or retinal lineage cells (i.e., it is a less developmentally potent cell in this context). Mesenchymal stem cells treated according to the methods described herein may in certain embodiments become more developmentally potent because they can readily differentiate into, for example, mesenchymal-lineage and neural-lineage cell types; the plasticity of the cells is increased when treated according to the methods of the invention.

The tissues formed in the devices of the invention will typically include one or more types of functional, mesenchymal or parenchymal cells, such as smooth or skeletal muscle cells, myocytes (muscle stem cells), fibroblasts, chondrocytes, adipocytes, fibromyoblasts, ectodermal cells, including ductile and skin cells, hepatocytes and other liver cells (e.g., Sinusoidal hepatic endothelial cells, Kupffer cells and hepatic stellate cells), kidney cells, pancreatic islet cells, cells present in the intestine, and other parenchymal cells, cells present in lung, osteoblasts and other cells forming bone or cartilage, and hematopoietic cells. In some cases it may also be desirable to include nerve cells. The vasculature will typically be formed from endothelial cells. “Parenchymal cells” include the functional elements of an organ, as distinguished from the framework or stroma. “Mesenchymal cells” include cells in connective and supporting tissues, smooth muscle, vascular endothelium and blood.

The devices may also be pre-seeded with an endothelial cell line to grow an endothelial layer on the outer or intraluminal (in certain embodiments) surfaces of the device prior to seeding the desired cells that ultimate form the three-dimensional tissue.

Cells can be obtained by biopsy or harvested from a living donor, cell culture, or autopsy, all techniques well known in the art. Cells are preferably autologous. Cells to be implanted can be dissociated using standard techniques such as digestion with a collagenase, trypsin or other protease solution and are then seeded into the mold or polymer scaffold immediately or after being maintained in culture. Cells can be normal or genetically engineered to provide additional or normal function. Immunologically inert cells, such as embryonic or fetal cells, stem cells, and cells genetically engineered to avoid the need for immunosuppression can also be used. Methods and drugs for immunosuppression are known to those skilled in the art of transplantation.

Undifferentiated or partially differentiated precursor cells may also be used. For example, the invention may use embryonic germ cells (Gearhart, et al., U.S. Pat. No. 6,245,566), embryonic stem cells (Thomson, U.S. Pat. Nos. 5,843,780 and 6,200,802), mesenchymal stem cells (Caplan, et al. U.S. Pat. No. 5,486,359), neural stem cells (Anderson, et al., U.S. Pat. No. 5,849,553), hematopoietic stem cells (Tsukamoto, U.S. Pat. No. 5,061,620), multipotent adult stem cells (Furcht, et al., WO 01/11011), all of are incorporated by reference. Cells can be kept in an undifferentiated state by co-culture with a fibroblast feeder layer (Thomson, U.S. Pat. Nos. 5,843,780 and 6,200,802), or by feeder-free culture with fibroblast conditioned media (Xu, et al. Nat. Biotechnol., 19, 971 (2001)). Undifferentiated or partially differentiated precursor cells can be induced down a particular developmental pathway by culture in medium containing growth factors or other cell-type specific induction factors or agents known in the art. Some examples of such factors include: vascular endothelial growth factor; Sonic Hedgehog; insulin-like growth factor II; osteogenin; cytotxic T cell differentiation factor; beta-catenin; bone morphogenic protein 2; interleukin 2; transforming growth factor beta; nerve growth factor; interleukin I; fibroblast growth factor 2; retinoic acid; and Wnt3.

A stem cell can be any known in the art, including, but not limited to, embryonic stem cells, adult stem cells, neural stem cells, muscle stem cells, hematopoietic stem cells, mesenchymal stem cells, peripheral blood stem cells and cardiac stem cells. Preferably, the stem cell is human. A “stem cell” is a pluripotent, multipotent or totipotent cell that can undergo self-renewing cell division to give rise to phenotypically and genotypically identical daughter cells for an indefinite time and can ultimately differentiate into at least one final cell type.

The quintessential stem cell is the embryonal stem cell (ES), as it has unlimited self-renewal and multipotent and/or pluripotent differentiation potential, thus possessing the capability of developing into any organ, tissue type or cell type. These cells can be derived from the inner cell mass of the blastocyst, or can be derived from the primordial germ cells from a post-implantation embryo (embryonal germ cells or EG cells). ES and EG cells have been derived from mice, and more recently also from non-human primates and humans. Evans et al. (1981) Nature 292:154-156; Matsui et al. (1991) Nature 353:750-2; Thomson et al. (1995) Proc. Natl. Acad. Sci. USA. 92:7844-8; Thomson et al. (1998) Science 282:1145-1147; and Shamblott et al. (1998) Proc. Natl. Acad. Sci. USA 95:13726-31.

The terms “stem cells,” “embryonic stem cells,” “adult stem cells,” “progenitor cells” and “progenitor cell populations” are to be understood as meaning in accordance with the present invention cells that can be derived from any source of adult tissue or organ and can replicate as undifferentiated or lineage committed cells and have the potential to differentiate into at least one, preferably multiple, cell lineages.

After the bioreactor devices of the invention are prepared, the devices themselves or a scaffold material (which may comprise the 124 polymer or elastomer thereof) integrated with the device (e.g., single wire scaffold, double wire scaffold, hollow tubular scaffold, three-dimensional scaffold with integrated channel / vascular system) can be seeded with the desired cells or sets of cells. Cells can be seeded onto the device or scaffold in an ordered manner using methods known in the art, for example, Teebken, et al., Eur J. Vasa Endovasc. Surg. 19, 381 (2000); Ranucci, et al., Biomaterials 21, 783 (2000). Also, tissue-engineered devices can be improved by seeding cells throughout the polymeric scaffolds and allowing the cells to proliferate in vitro for a predetermined amount of time before implantation, using the methods of Burg et al., J. Biomed. Mater. Res 51, 642 (2000).

For purposes of this invention, “animal cells” can comprise endothelial cells, parenchymal cells, bone marrow cells, hematopoietic cells, muscle cells, osteoblasts, stem cells, mesenchymal cells, sembryonic stem cells, or fibroblasts. Parenchymal cells can be derived from any organ, including heart, liver, pancreas, intestine, brain, kidney, reproductive tissue, lung, muscle, bone marrow or stem cells.

In one embodiment, the mold or polymer scaffold is first seeded with a layer of parenchymal cells, such as hepatocytes or proximal tubule cells, or endothelial cells. This layer can be maintained in culture for a period of time, e.g., a week or so, in order to obtain a population doubling. It can be maintained in a perfusion bioreactor to ensure adequate oxygen supply to the cells in the interior.

Sets of cells can be added to or seeded into the three-dimensional apparatuses/devices of the invention, which can serve as a template for cell adhesion and growth by the added or seeded cells. The added or seeded cells can be parenchymal cells, such as hepatocytes or proximal tubule cells. Stem cells can also be used. A second set of cells, such as endothelial cells, can be added to or seeded onto the assembled apparatus through other vessels than those used to seed the first set of cells. The cell seeding is performed by slow flow. As a practical matter, the geometry of the apparatus will determine the flow rates. In general, endothelial cells can enter and form vessel walls in micromachined channels that are about 10-50 μm. Thus, in addition to serving as a mechanical framework for the organ, the assembled apparatus provides a template for all of the microstructural complexity of the organ, so that cells have a mechanical map to locate themselves and form subsystems, such as blood vessels in the liver.

Molecules such as growth factors or hormones can be physically trapped or covalently attached to the surface (e.g., by absorption) of the devices/scaffolds to effect growth, division, differentiation or maturation of cells cultured thereon. In other embodiments, the devices/scaffolds of the herein disclosed bioreactor systems may include materials such as growth factor and hormones and other suitable tissue culture agents integrated (e.g., covalent or non-covalently interactions) directly into the polymers that comprise the devices/scaffolds, e.g., forming covalent or non-covalent bonds with the polymer materials, which could be added during bulk processing of the polymers.

### Applications

Three-dimensional tissue systems of the invention comprising scaffolds of the 124 polymer or elastomers thereof are useful for a variety of applications, including but not limited to, (a) the testing of the efficacy and safety (including toxicity) of experimental pharmacologic agents (including, but not limited to, small molecule drugs, biologics, nucleic acid-based agents), (b) the defining of pharmacokinetics and/or pharmacodynamics of pharmacologic agents (including, but not limited to, small molecule drugs, biologics, nucleic acid-based agents), (c) characterizing the properties and therapeutic effects of pharmacologic agents (including, but not limited to, small molecule drugs, biologics, nucleic acid-based agents) on a subject, (d) screening of new pharmacologic agents, (e) providing implantable engineered tissues for use in regenerative medicine for treating damaged and/or diseased tissues, (e.g., use of the tissue constructs, devices, and/or systems of the disclosure to study cardiac disease states, including patients with electrical conduction defects (iPSC-CM)), and (f) personalized medicine.

In certain embodiments, the three-dimensional tissue systems of the invention may be suitable for cultivation and generation of various tissue structures. The disclosed devices may be designed to provide an in vitro platform that mimics or reproduces native tissue architecture found in vivo, to enable cells to mature and function in the way they normally would in vivo.

In other embodiments, the disclosed devices may be suitable for culture of various tissues, including muscle cells such as cardiomyocytes, skeletal muscle cells, smooth muscle cells as well as excitable tissues such as neurons and cells that may require rich vasculature such as hepatocytes, among others.

In still other embodiments, the disclosed devices may be suitable for various applications, including drug-testing in vitro, for building a human-on-a-chip with several different compartments as well as for direct anastomosis and implantation into an animal or a human patient, among other applications.

In certain embodiments, the three-dimensional tissue engineered systems of the invention can be used to measure the metabolism, toxicity and efficacy of test agents. Methods of the invention can be used to screen experimental drugs or “test agents” that have no known metabolic or pharmacokinetic profile, in order to obtain such information, including information necessary to assess toxicity. Toxicity can often occur as a result of drug-to-drug interactions. Thus, methods of the invention can be used to study the combination of test agents with known drugs or other test agents. These methods are particularly relevant to use in clinical settings since many patients are treated with multiple drugs.

In general, test agents can be incubated with the three-dimensional tissue engineered systems of the invention in a dosage range estimated to be therapeutic and for a duration sufficient to produce an effect (e.g., metabolic effects or effects indicating to toxicity or efficacy). The incubation time can range between about 1 hour to 24 hours, or can be extended as necessary for several days or even weeks. The incubation conditions typically involve standard culture conditions known in the art, including culture temperatures of about 37 degrees Celsius, and culture mediums compatible with the particular cell type selected.

Test agents that can be analyzed according to methods of the invention include, but are not limited to, opioid analgesics, anti-inflammatory drugs such as antihistamines and non-steroidal anti-inflammatory drugs (NSAIDs), diuretics such as carbonic anhydrase inhibitors, loop diuretics, high-ceiling diuretics, thiazide and thiazide-like agents, and potassium-sparing diuretics, agents that impinge on the renal and cardiovascular systems such as angiotensin converting enzyme (ACE) inhibitors, cardiac drugs such as organic nitrates, calcium channel blockers, sympatholytic agents, vasodilators, .beta.-adrenergic receptor agonists and antagonists, .alpha.-adrenergic receptor agonists and antagonists, cardiac glycosides, anti-arrhythmic drugs, agents that affect hyperlipoproteinemias such as 3-hydroxymethylglutaryl-coenzyme A (HMG-CoA) inhibitors, anti-neoplastic agents such as alkylating agents, antimetabolites, natural products, antibiotics, and other drugs, immunomodulators, anti-diabetic agents, and anti-microbial agents such as antibacterial agents, antiviral agents, antifungal agents, antiprotozoal agents, and antihelminthic agents, but are not limited to these agents.

For example, the three-dimensional tissue systems of the invention can be used to detect/evaluate toxicity associated with therapeutic agents (e.g., cardio-toxicity, or liver toxicity associated with drug administration). There are three general classes of toxicity. Acute toxicity is a toxic effect that occurs after less than about 24 hours of exposure to the drug. Subacute toxicity occurs later, after about 14 to 90 days of exposure to the drug. Chronic toxicity occurs after about 90 days (or longer) exposure to the drug. Current methods in the art are suboptimal for use in detecting subacute and chronic toxicity due to the requirement for extended periods of monitoring in a living subject. While methods of the invention can encompass these longer intervals of exposure, effects may be detected more rapidly, such that the incubation time for the test agent need not be extended. Accordingly, incubation times can range between about 1 hour to 24 hours, or can be extended as necessary for several days or even weeks.

The undesired effects of toxicity caused by administration of a test agent can be screened in several ways. Tissue engineered systems of the invention can be used to determine the range of toxic dosimetry of a test agent. The effect of increasing concentrations of the test agent (i.e., dose) on tissues of interest can be monitored to detect toxicity. A toxic effect, when observed, can be equated with a measurement of test agent concentration/cells cm². By calculating the toxic concentration according to the distribution of cells in the tissue engineered system, one of skill in the art can extrapolate to the living system, to estimate toxic doses in subjects of various weights and stages in development.

Using methods of the present invention, various doses of individual test agents and combinations of test agents with other pharmaceuticals will be screened to detect toxic effects, including but not limited to irregular metabolism, cardiotoxicity, liver toxicity, carcinogenicity, kidney and neural toxicity and cell death. To detect irregular changes in metabolism, standard methods known in the art for assaying metabolite production, including but not limited to glucose metabolism and enzymatic assays, can be employed. The particular metabolic pathway assayed, or metabolite measured, can vary according to the tissue type selected.

In detecting carcinogenicity, cells can be screened for a transformed phenotype using methods well known in the art, for example, methods detecting changes in gene expression, protein levels, abnormal cell cycles resulting in proliferation and changes in expression of cell surface markers, including, but not limited to, antigenic determinants. Gene expression patterns can be determined, for example, by evaluating mRNA levels of genes of interest according to standard hybridization techniques, such as RT-PCR, in situ hybridization, and fluorescence in situ hybridization (FISH), Northern analysis or microchip-based analysis. Protein expression patterns can be determined by any methods known in the art, for example, by quantitative Western blot, immunohistochemistry, immunofluorescence, and enzyme-linked immunosorbent assay (ELISA), amino acid sequence analysis, and/or protein concentration assays. For details, see Sambrook, Fritsch and Maniatis, Molecular Cloning, A Laboratory Manual, 2nd Ed., Cold Spring Harbor Laboratory Press, 1989. Cell counting and/or separation techniques, such as FACS analysis, can be employed to measure proliferation or detect aberrant cell surface marker expression.

Standard methods well known in the art can also be used to detect cell death, including but not limited to, tunnel assays. Traditional approaches of in vitro toxicology to toxicological screening has been to measure comparatively late events in the process of cell death, such as lactate dehydrogenase release or differential counting of viable and dead cells using vital dyes, such as trypan blue, 4,6-diaminophenylindole (DAPI), propidium iodide, and LIVE/DEAD stain available from Molecular Probes. Prediction of lethality in vivo is one proposed application of this type of in vitro screen, although cell death is not a common mechanism by which the animal's death is induced following acute exposure to a toxic agent. In contrast, caspase activation is at the center the common features of chronic toxicity, cell death, hyperproliferation and inflammatory reactions. Caspase activity can be measured relatively quickly after a toxic insult (30 min to 4 hr) by fluorescence spectroscopy, thus lending itself to high-throughput screening techniques. Other markers and assays commonly used to monitor apoptosis or necrosis of cells can include, but are not limited to, the presence of phosphatidylserine on the outer leaflet of the plasma membrane of affected cells, annexin V staining, and terminal deoxynucleotidyltransferase nick-end labeling assay (TUNEL).

Using methods of the invention, various doses of individual test agents and combinations of test agents will be screened in panels comprised of tissues having diverse genetic backgrounds to determine the pharmacogenetic toxicity profile of the test agents. For example, multiple doses of, or combinations with, test agents will be screened for toxic effects specific to one or more genetic backgrounds. Toxic effects to be screened for genetic variance include, but are not limited to, irregular metabolism, carcinogenicity and cell death.

Tissue-engineered devices of the present invention can be modified in parallel to generate a comprehensive array of the currently known genetic polymorphisms of different metabolic enzymes. A salient example is the CYP450 monooxygenase system, wherein the population comprises multiple isoforms and polymorphisms that impinge on and complicate predictive models of drug metabolism, drug clearance, and toxicity. For example, in the metabolism of thiopurines, such as thioguanine, the rate-limiting enzyme is a methyltransferase that has different polymorphic forms. Polymorphism in the methyltransferases is known to affect metabolism of the thiopurines. Where the polymorphism gives rise to slower metabolism of the thiopurine, clinical benefit is decreased and where the polymorphism gives rise to an increased rate of metabolism, toxicity can result. Thus, methods of the invention can be used to determine the metabolic profile of various test agents in the presence of various polymorphic forms of an enzyme, such as methyltransferase.

In testing for differential toxicity due to polymorphic variation, or other genetic defects, genetically engineered cells comprising gene knockouts or knock-ins of specific enzymes known to affect drug metabolism and toxicity can be used in the systems of the invention. Cells can be modified using techniques that are known to the skilled artisan, such as RNA interference (RNAi), antisense technology, ribozymes, site-directed mutagenesis, among others.

When evaluating effects on metabolism, levels of metabolites, if known, can be detected using methods well known in the art as a reflection of metabolic activity, such as liquid chromatography. Liquid chromatography coupled with tandem mass spectrometric detection (LC/MS/MS) can be used as an analytical method to monitor early absorption, distribution, metabolism and elimination testing. This method provides excellent sensitivity, specificity and high sample throughput. The quantitative selectivity afforded by reaction monitoring on a triple quadrupole instrument precludes the need for high chromatographic resolution or extensive sample clean up. Using automated sample-processing techniques, such as on-line column switching, combined with high-sample-density microtiter plates, can further maximize analytical throughput. Modern LC/MS/MS also offers limits of detection extending down to the sub-nanogram per ml range using only minimal quantities of biological matrix.

LC/MS/MS enables rapid and sensitive quantitation of new drug candidates, as well as providing important structural information on metabolites. A full scan LC/MS analysis can initially suggest possible oxidative and/or conjugative metabolic transformations on the basis of the ionic species observed. In the MS/MS mode, the instrument can be tuned to a selected precursor ion of interest, which is then further fragmented to form productions that uniquely identify the metabolic (production scan).

Selectivity can be further enhanced by the quadrupole ion trap, a device that “traps” ions in a space bounded by a series of electrodes. The unique feature of the ion trap is that an MS/MS experiment (or, in fact, multi-step MS experiments) can be performed sequentially in time within a single mass analyzer, yielding a wealth of structural information. Hybrid quadrupole-time-of-flight (Q-TOF) LC/MS/MS systems can also be used for the characterization of metabolite profiles. The configuration of Q-TOF results in high sensitivity in mass resolution and mass accuracy in a variety of scan modes.

Liquid chromatography coupled with nuclear magnetic resonance spectroscopy (LC-NMR) provides a way of confirming absolute molecular configurations. A linear ion-trap mass spectrometer possesses significantly enhanced production-scanning capabilities, while retaining all of the scan functions of a triple quadrupole MS. The ultra-high resolution and sensitivity of Fourier transform ion-cyclotron resonance MS (FI-ICRMS) can be useful for the analysis and characterization of biological mixtures. Data processing and interpretation software packages also enable efficient identification and quantification of metabolites using the tissue-engineered devices of the present invention.

A widely used method to study in vitro drug metabolism is the use of tissue homogenates. The tissues within the three-dimensional systems of the invention can be cultured in the presence of a test agent and harvested to obtain tissue homogenate preparations for use in enzyme analysis. Preparation of tissue homogenates is well known in the art and involves the steps of tissue homogenization and subcellular fractionation to yield two main fractions routinely studied in drug metabolism: the post-mitochondrial supernatant and the endoplasmic reticulum (microsomal) fraction.

The three-dimensional tissue culture devices of the invention can also be used to evaluate a test agent's efficacy. Efficacy can be detected by measuring individual parameters associated with the repair, enhancement, improvement and/or regeneration of a disease model comprising an injured tissue grown in a three-dimensional system of the invention. In disease models of the invention, the injury can be induced or can be the result of a pre-existing condition in the tissue donor, including conditions relating to inherited genetic abnormalities. Either the induced or pre-existing condition can comprise a weakened state resulting from a previous drug exposure. Test agents, or combinations of test agents, can be analyzed for efficacy in disease models of the invention.

In one embodiment, selected tissues of interest can be treated with agents known in the art to cause cellular damage (e.g., toxins, mutagens, radiation, infectious agents and chemical agents), inducing injury in the tissue. In another embodiment, selected tissues of interest can be altered using standard recombinant techniques to induce a disease state. For example, techniques of homologous recombination can be used to insert a transgene into a cell, or “knock-out” gene expression of a gene of interest. For a review of homologous recombination, see Lewin, B., Genes V, Oxford University Press, New York, 1994, pp. 968-997; and Capecchi, M., (1989) Science 244:1288-1292; Capecchi, M., (1989) Trends Genet. 5 (3):70-76. In another embodiment, the selected tissue of interest is injured as a result of an inherited genetic defect, which can be a single gene defect or a multifactorial defect. For a discussion of inherited disorders, see Thompson, McInnes and Willard, Genetics in Medicine, 5.sup.th Ed., W.B. Saunders Company, 1991.

Tissue engineered systems of the invention can be used to determine the range of effective dosimetry of a test agent. The effect of increasing concentrations of the test agent (i.e., dose) on tissues of interest can be monitored to detect efficacy. A therapeutic effect, when observed can be equated with a measurement of concentration/cells cm². By calculating the effective concentration according to the distribution of cells in the tissue engineered system, one of skill in the art can extrapolate to the living system, to estimate therapeutic doses in subjects of various weights.

Using methods of the invention, various doses of individual test agents and combinations of test agents will be screened in panels comprised of tissues having diverse genetic backgrounds to determine the pharmacogenetic efficacy profile of the test agents. For example, multiple doses of, or combinations with, test agents will be screened for efficacy, or the lack thereof, specific to one or more genetic backgrounds.

Methods of the invention can be carried out using tissues of any kind. The following description provides specific information relating to three preferred embodiments of the invention, which include the use of the systems/devices of the invention with cells derived from liver, heart, and kidney.

### Liver

The liver plays a major role in carbohydrate metabolism by removing glucose from the blood, under the influence of the hormone insulin, and storing it as glycogen. When the level of glucose in the blood falls, the hormone glucagon causes the liver to break down glycogen and release glucose into the blood. The liver also plays an important role in protein metabolism, primarily through deamination of amino acids, as well as the conversion of the resulting toxic ammonia into urea, which can be excreted by the kidneys. In addition, the liver participates in lipid metabolism by storing triglycerides, breaking down fatty acids, and synthesizing lipoproteins. The liver also secretes bile, which helps in the digestion of fats, cholesterol, phospholipids, and lipoproteins.

Analysis of metabolic function will indicate toxicity in liver. Thus, in liver tissue engineered systems of the invention, metabolic assays to detect toxicity of a particular test agent are preferred. Metabolic enzymes, including but not limited to, cytochrome P450, alkaline phosphatase, glycolytic enzymes such as alpha-galactosidase, beta-galactosidase, alpha-glucosidase, beta-glucosidase, alpha-glucuronidase, beta-glucuronidase, and alpha-amylase, NADPH-cytochrome P450 reductase, cytochrome b₅, N-demethylase, O-demethylase, acetylcholinesterase, pseudocholinesterase, among other esterases, epoxide hydrolase, amidases, Uridine diphosphate (UDP)-glucuronosyltransferases, phenol sulfotransferase, alcohol sulfotransferase, sterid sulfotransferase, and arylamine sulfotransferase, UDP-glycosyltransferases, purine phosphoribosyltransferase, N-acetyltransferases, glutathione S-transferase, phenylethanolamine N-methyltransferase, non-specific N-methyltransferase, imidazole N-methyltransferase, catechol-O-methyltransferase, hydroxyindole-O-methyltransferase, and S-methyltransferase, alcohol dehydrogenase, aldehyde dehydrogenase, xanthine oxidase, amine oxidases such as monoamine oxidases, diamine oxidases, flavoprotein N-oxidases, and hydroxylases, aromatases, cysteine conjugate .beta.-lyase, and alkylhydrazine oxidase can be tested for metabolic activity using assays well known in the art (this is described in great detail in other portions of the application). Cytochrome p450 enzymes that can be tested include, but are not limited to, CYP1A1, CYP1A2, CYP2A3, CYP2B6, CYP2B7, CYP2B8, CYP2C8, CYP2C9, CYP2C10, CYP2D6, CYP2D7, CYP2D8, CYP2E1, CYP2F1, CYP3A3, CYP3A4, CYP3A5, and CYP4B1.

In one embodiment, the test agent comprises antiviral activity, most preferably, antiviral activity against hepatitis. Currently, there is a great need for safe and effective treatments for hepatitis (Mutchnick, M. G., et. al., Antiviral Research (1994) 24:245-257). For example, clinical tests on the use of the nucleoside analog fialuridine (FIAU) for treatment of chronic hepatitis B were suspended recently due to drug-related liver failure leading to death in some patients. Test agents demonstrating efficacy against hepatitis can also be screened for acute, subacute and chronic toxicity by monitoring metabolic function, preferably of metabolic function of cytochrome P450 and alkaline phosphatase, following administration.

Test agents can be screened for efficacy in tissue engineered systems of the invention comprising liver cells affected with diseases including, but not limited to, cancer, diabetes, acute hepatitis, fulminant hepatitis, chronic hepatitis, hepatic cirrhosis, fatty liver, alcoholic hepatopathy, drug induced hepatopathy (drug addiction hepatitis), congestive hepatitis, autoimmune hepatitis, primary biliary cirrhosis and hepatic porphyria, and pericholangitis, sclerosing cholangitis, hepatic fibrosis and chronic active hepatitis, which have been reported to occur with a high frequency as complications of inflammatory bowel diseases such as ulcerative colitis and Crohn's disease.

Preferably, test agents will be assayed for their ability to reduce or prevent of progress of hepatic necrocytosis and/or accelerate hepatic regeneration. For example, expression levels of Rasp-1, a gene that is upregulated during regeneration of liver tissue, can be monitored following administration of a test agent. Rasp-1 is described in U.S. Pat. No. 6,027,935, the contents of which are incorporated herein by reference for their description of Rasp-1 sequences, antibodies and assays.

In a preferred embodiment, test agents are screened for efficacy in the treatment of hepatitis viral infections, particularly infections of hepatitis B and hepatitis C. Other hepatitis viruses that are significant as agents of human disease include hepatitis A, hepatitis delta, hepatitis E, hepatitis F, and hepatitis G (Coates, J. A. V., et. al., Exp. Opin. Ther. Patents (1995) 5 (8): 747-756). The test agent can comprise, for example, nucleoside analog antivirals, immunomodulators, immunostimulators (e.g., interferons and other cytokines) or other immune system-affecting drug candidates, including, but not limited to, thymic peptides, isoprinosine, steroids, Schiff base-forming salicylaldehyde derivatives such as Tucaresol, levamisol, and the like (Gish, R. G., et al., Exp. Opin. Invest. Drugs (1995) 4 (2):95-115; Coates, J. A. V., et al., Exp. Opin. Ther. Patents (1995) 5 (8):747-765).

Anti-hepatitis efficacy of a test agent can be determined according to methods known in the art. For example, following treatment with a test agent, the amount of hepatitis virus or viral DNA in the culture medium can be determined by PCR analysis (e.g., of sedimented particles). DNA measurements can be correlated with viral replication to assess post-treatment infectivity. Alternatively, viral loads can be measured directly. Other measures of efficacy include measurement of enzyme levels, including but not limited to SGOT, ALT and LDH, histologic analysis and normal production of total liver proteins, such as the clotting factors.

In a preferred embodiment, the efficacy of a test agent is determined in liver tissues infected with the hepatitis C virus. In a preferred embodiment, test agents are screened for efficacy in the treatment of liver cancer. Reduction or elimination of transformed liver cells in response to treatment with a test agent can be detected by measuring decreases in hypercalcaemia and CEA expression. Reduction in proliferation can also be determined by cell counting.

### Heart

The toxic effect of a test agent in cardiac tissue engineered systems of the invention can be detected using a variety of assays known in the art. The tissue culture systems of the invention that comprise scaffolds comprising the 124 polymer or elastomers thereof are particularly suited for cardiac tissue applications since the mechanical properties and elasticity of the elastomers of the invention are similar to adult cardiomyocytes.

Assays to detect toxicity of a particular test agent preferably comprise measurement of QT intervals, changes in electrophysiology (e.g., changes in K⁺/Ca²⁺ channels, hERG) and/or arrhythmia by T-wave alternans (TWA).

Alternans of the electrocardiogram is defined as a change in amplitude and/or morphology of a component of the ECG that occurs on an every-other-beat basis (Walker, M. L. and Rosenbaum, D. S., (2003) Cardiovasc. Res. 57: 599-614). TWA is the beat-to-beat alternation of T-wave amplitude, and is closely linked to electrical instability in the heart. Beat-to-beat microvolt fluctuation of the T wave can be detected using high-resolution electrodes and signal processing techniques (Gold, M. R., and Spencer, W. (2003) Curr. Opin. Cardiol. 18: 1-5). A large number of beats, generally 128, are sampled, and the voltages of multiple corresponding points on the T-wave are computed and averaged. Through fast-Fourier transformation, these consecutive amplitudes are displayed spectrally, yielding several frequency peaks. These peaks correspond to thoracic excursions with respiration, other repetitive body movements, and ambient electrical noise. The peak at 0.5 cycles/beat, if present, is caused by TWA. The alternans magnitude, Vₐₗₜ, represents the difference between the even or odd beat and the mean amplitude, in microvolts. A threshold of 1.9 uV is used for significance. The alternans ratio (k) is another parameter measured and represents the ratio of the alternans amplitude to the SD of the background noise. It is required to be greater than 3.0 for significance. Additionally, TWA must be sustained for more than one minute.

Test agents can be screened for efficacy in tissue engineered systems of the invention comprising cardiac cells affected with diseases including, but not limited to, congestive heart failure, coronary artery disease, myocardial infarction, myocardial ischemia, effects of atherosclerosis or hypertension, cardiomyopathy, cardiac arrhythmias, muscular dystrophy, muscle mass abnormalities, muscle degeneration, myasthenia gravis, infective myocarditis, drug- and toxin-induced muscle abnormalities, hypersensitivity myocarditis, autoimmune endocarditis, and congenital heart disease. Preferably, test agents will be assayed for their ability to accelerate cardiac regeneration or improve contractile properties. In general, efficacy can be indicated by detection of improved contractility, electromechanical conduction and/or association, susceptibility to electrical dysfunction, ventricular fibrillation (sudden death), ionotropy, chronotropy, and decreased leakage of enzymes (e.g., CPK and SGOT).

In various embodiments, the devices of the invention can be utilized or coupled together, including, e.g., in series (i.e., in tandem), in parallel or combinations thereof, wherein a first device prepared from a first type of tissue (e.g., hepatic) can be linked in series with a second device prepared from a second type of tissue (e.g., cardiac) in order to study drug effects and interactions with multiple tissues. For example, a plurality of Angiochip or Angiotube sytems may be configured in series, whereby a first Angiochip/Angiotube is formed of one type of cell or tissue (e.g., cardiac) and a second “downstream” or “upstream” Angiochip/Angiotube is formed of a second type of cell or tissue (e.g., diseased cardiac, or hepatic). In this manner, the interaction of drugs may be tested in the context of multiple organ or tissue sytems. For example, a test agent may be introduced into an Angiochip/Angiotube prepared from hepatic tissue, which may be linked downstream to a second Angiochip/Angiotube prepared from cardiac tissue. In this manner, the drug may first interact with the hepatic tissue, and any metabolic products resulting therefrom may flow downstream to the cardiac tissue Angiochip/Angiotube, thereby facilitating one to test the effect of the drug's metabolism on cardiac function. Thus, the invention contemplates a plurality of devices arranged in a tandem (i.e., in series) manner for use in testing inter-organ drug interactions in the body. Any conceivable combination of tissues could be tested in tandem, for example, cardiac/hepatic or hepatic/cardiac.

### Kidney

Pharmaceuticals and biologics are a common source of kidney injury (i.e., nephrotoxicity), causing approximately 20% of acquired episodes of acute renal failure (ARF). The development of acute renal failure (ARF) in a hospitalized patient results in a 5-fold to 8-fold higher risk of death. Although hemodialysis, hemofiltration and peritoneal dialysis treatment with its small solute and fluid clearance function has prevented death from hyperkalemia, volume overload and uremic complications, such as pericarditis, patients with ARF still have mortality rates exceeding 50. It is not a complete renal replacement therapy because it only provides filtration function and does not replace the hemostatic, regulatory, metabolic, and endocrine function. Patients with end stage renal disease on dialysis continue to have major medical, social and economic problems. Most drugs found to cause nephrotoxicity exert toxic effects by one or more common pathogenic mechanisms. These include altered intraglomerular hemodynamics, tubular cell toxicity, inflammation, crystal nephropathy, rhabdomyolysis, and thrombotic microan-giopathy. Knowledge of offending drugs and their particular pathogenic mechanisms of renal injury is critical to recognizing and preventing drug-induced renal impairment. A safer and more effective assay for measuring the potential for nephrotoxicity of drugs and biologics would no doubt significantly help reduce the amount of kidney injury today due to medication. Thus, in certain embodiments the bioreactor systems described herein, including, but not limited to a biowire system, a biotube system, a biorod system, an angiochip system, or an antiotube system, can be used to assess kidney tissue, and in particular, measure or assess nephrotoxic effects of drugs and biologics on the kidney.

The methods and bioreactor systems described herein, including, but not limited a biowire system, a biotube system, a biorod system, an angiochip system, or an antiotube system, can be used to test any drug or biologic of interest. Such agents can include active agents known to be nephrotoxic, such as radiographic contrast media (e.g., “contrast agent” or “dye”), non-steroidal anti-inflammatory drugs (NSAID's), amphotericin, cisplatin, methotrexate, acyclovir, gentamicin, acetylcholinesterase inhibitiors, other nephrotoxic drugs, and internally generated substances such as products of tumor lysis and products of rhabdomyolysis and toxins associated with infections or septicemia, and the methods of the present invention may also be used to prevent or mitigate renal damage due to an overdose or other ingestion, absorption or exposure to such nephrotoxic substances.

In various embodiments, the devices of the invention can be utilized or coupled together, including, e.g., in series (i.e., in tandem), in parallel or combinations thereof, wherein a first device prepared from a first type of tissue (e.g., renal) can be linked in series with a second device prepared from a second type of tissue (e.g., cardiac) in order to study drug effects and interactions with multiple tissues. For example, a plurality of Angiochip or Angiotube sytems may be configured in series, whereby a first Angiochip/Angiotube is formed of one type of cell or tissue (e.g., cardiac) and a second “downstream” or “upstream” Angiochip/Angiotube is formed of a second type of cell or tissue (e.g., diseased cardiac, or renal). In this manner, the interaction of drugs may be tested in the context of multiple organ or tissue sytems. For example, a test agent may be introduced into an Angiochip/Angiotube prepared from hepatic tissue, which may be linked downstream to a second Angiochip/Angiotube prepared from renal tissue. In this manner, the drug may first interact with the hepatic tissue, and any metabolic products resulting therefrom may flow downstream to the renal tissue Angiochip/Angiotube, thereby facilitating one to test the effect of the drug's metabolism on organ function. Thus, the invention contemplates a plurality of devices arranged in a tandem (i.e., in series) manner for use in testing inter-organ drug interactions in the body. Any conceivable combination of tissues could be tested in tandem, for example, renal/hepatic or hepatic/renal.

### EXAMPLES

### Example 1

### Preparation of a Highly Elastic and Moldable Polyester Biomaterial for Cardiac Tissue Engineering Applications

### Introduction

Engineered tissue constructs rely on biomaterial polymers as support structures for tissue

Construction. These materials form a support mechanism that assist immature groups of cells

to develop into complex tissue networks that exhibit the properties of native cells, and support

the integration of these complexes into the host surroundings. It is important that these

polymers mimic the physical properties of the host tissue, have appropriate degradation

properties, and limit host response.

Recently, the tissue engineering community has increasingly focused on the utilization of polyester biomaterials for scaffold construction. These polymers are desirable for their simple synthesis procedure, hydrolytic degradation properties, and elastomeric characteristics. There has been a number of notable applications of polyester materials in FDA-approved products, including polycapralactone and poly-L-lactic acid, but the high stiffness of these materials limits their use in soft tissue scaffolds. These limitations have directed synthesis efforts towards developing materials exhibiting more elastic properties.

Of particular note, biomaterial-based cardiac tissue engineering solutions rely on optimized material properties which mimic the properties of native cardiac tissue for effective development of tissue constructs. Many criteria must be considered including elasticity, degradation rate, and material compatibility in vivo.

Materials used for cardiac tissue engineering are difficult to optimize. Ventricular filling and ejection leads to repetitive cyclic loading on the material construct which they must withstand while ensuring they do not constrict the tissue to which support is being provided. Therefore, matching the mechanical properties of the heart is important. The human heart has a Young's Modulus that varies from 10-20 kPa at the beginning of diastole to 200-300 kPa at the end of systole. Immature engineered cardiac tissue constructs tend to exhibit properties on the low end of this range, so a more elastic material is desirable to ensure tissue maturation is not inhibited.

It is important that properties match this elasticity, as those that are too stiff can be found to restrict tissue contraction. Therefore, an ideal biodegradable elastomer for cardiac tissue engineering should exhibit a relatively low Young's modulus, with high elongation and tensile strength. Furthermore, the material should exhibit degradation properties that allow the breakdown of the scaffold in vivo over a 6-8 week period to support engineered tissue integration with the host but not restrict continued regeneration. Finally, materials must be compatible with the immune system of the host, as inflammatory and immune response to tissue engineering constructs greatly limits their regenerative potential. An ideal polymer should be an elastomer constructed of biocompatible monomers and prepared via a simple synthetic route.

In this Example, a two-step elastomer preparation process is presented which incorporates photocuring moieties to develop a final crosslinked structure. This enables molding of a pre-polymer gel into complex shapes needed for cardiac tissue engineering, where the final function of the tissue is strongly determined by its structure.

Furthermore, the data shows that the elastomeric properties of the elastomer mimicked those of human adult myocardiam, and the material exhibited characteristics for appropriate use both in vitro and in vivo.

Accordingly, this Example describes an approach for rational design of cardiac tissue engineering elastomers, results of their toxicity testing followed by a full characterization of the of an exemplary elastomer material prepared from crosslinked monomers of poly(octamethylene maleate (anhydride) 1,2,4-butanetricarboxylate) (i.e., the 124 polymer).

This material was synthesized through a one-step polycondensation of 1,8-octanediol,

1,2,4-butanetricarboxylic acid and maleic anhydride. The Example provides a characterization of its mechanical properties, degradation rate and cell compatibility both in vitro and in vivo.

### Synthesis of the 124 Pre-Polymer

Poly(octamethylene maleate (anhydride) 1,2,4-butanetricarboxylate) (124 polymer) can be achieved by first synthesizing polyester pre-polymer through combining 1,8-octanediol (e.g., obtainable commercially from SIGMA®), maleic anhydride (e.g., obtainable commercially from SIGMA®), and 1,2,4-butanetricarboxylic acid (e.g., obtainable commercially from SIGMA®) monomers in a 250 mL round bottom flask under nitrogen flow; the ratio of hydroxyl to carboxylic acid end groups of the monomers were kept at a 1:1 ratio to allow for complete reaction of chemically active sites while the ratio of 1,2,4-butanetricarboxylic acid to maleic anhydride was varied; the monomers was melted at 150° C. with stirring at 200 rpm until the viscosity increased to a point that the magnetic stirring bar was immobilized. The crude polymer solution was then dissolved in 1,4-dioxane (e.g., obtainable commercially from SIGMA®) and dripped into deionized distilled water. The liquid mixture was decanted and the purified polymer was collected and dried under constant air flow for 48 hours.

### Photocrosslinking of 124 Polymer

The 124 pre-polymer was crosslinked by free radical polymerization to form the 124 polymer, an elastomer. The purified pre-polymer was dissolved in dimethylsulfoxide (DMSO) . The pre-polymer solution was mixed with 5 wt % UV initiator (2-hydroxy-1-[4(hydroxyethoxy)phenyl]-2-methyl-1 propanone, Irgacure 2959, e.g., obtainable commercially from SIGMA®) by heating the mixture above 100° C. and allowing the initiator powder to fully mix in the polymer solution.

In one embodiment, poly(ethylene glycol) dimethyl ether (PEGDM, SIGMA®) was added as a porogen to the solution at the desired concentration (wt %). The poragen was leached out in Dulbecco's Phosphate Buffered Saline (PBS, Gibco) post UV crosslinking to make a nanoporous structure.

It should be noted that failed polymer combinations using tartaric and malic acid were synthesized using this procedure with the substitution of 1,2,4-butanetricarboxylic acid with the respective carboxylic acid monomer.

### POMaC Pre-Polymer Synthesis

POMaC pre-polymer was synthesized by first combining the melted 1,8-octanediol, maleic anhydride, and citric acid (CALEDON®) under nitrogen flow. Polycondensation was then carried out under stirring at elevated temperature to obtain a purified concentrated pre-polymer. Final elastomeric structures were developed with mixing of pre-polymer gel with UV initiator and exposure to UV light according to application. When appropriate, nanoporous structures were developed with mixing of poragen (or “porogen”) with the pre-polymer gel. As the skilled person will understand, a poragen any variety of particles having a specified shape and size that is used to make pores in molded structures in tissue engineering. The particles typically dissolve away or are leached out after the structures have set. Characterization of polymer properties

Polymer structure was confirmed using ¹H NMR on an Agilent DD2 600 MHz spectrometer. Polymer samples were dissolved in deuterated dimethyl sulfoxide (DMSO-d₆, SIGMA®). Chemical shifts were tested against the resonance of protons in internal tetramethylsilane (TMS).

Contact angle was measured by sessile drop method (deionized water) in air using a Goniometer (Rame-hart Model 100-00) modified with a digital microscope incorporated for image acquisition (resolution 640x480 pixels grayscale) and a Rame-hart HPLC straight needle, size 22G, attached to a 2 mL Gilmont microsyringe. Angles were manually measured on both sides of the drop and averaged.

### Neonatal Rat Heart Cell Isolation

Neonatal (1-2 day old) Sprague-Dawley rats (Charles River) were euthanized, and excised hearts were collected in Ca²⁺, Mg²⁺ free Hank's balanced salt solution (HBSS) (GIBCO®). The aorta and vena cava structures were removed from the hearts, which were then quartered. The heart sections were rinsed twice in ice cold HBSS, then digested overnight (4° C.) in of a solution 0.06% (w/v) trypsin (SIGMA®) in HBSS. Further digestion was done using collagenase II (Worthington, 200 U/ml) in HBSS (37° C.) in five sets of 4-8 min digestions. Following digestion, cells were preplated for 40 min and the nonadherent cells were utilized as the rat cardiomyocyte (CM) population. Rat CMs were cultured in Dulbecco's modified Eagle's medium (GIBCO®) containing glucose (4.5 g/liter), 10% (v/v) fetal bovine serum (FBS; GIBCO®),1% (v/v) Hepes (100 U/ml; GIBCO®), and 1% (v/v) penicillin-streptomycin (100 mg/ml; GIBCO®).

### Mechanical Characterization

The 124 polymer Young's Modulus was characterized through tensile testing. A three-factor design of experiment (DOE) was conducted to evaluate the range of modulus that 124 polymer can achieve as a result of variations in the preparation procedure. The three factors considered were: (A) monomer feed ratio of 1,2,4-butanetricarboxylic acid and maleic anhydride (124:MA); (B) UV exposure energy dose (mJ) and (C) porous fraction, achieved by the incorporation of porogen into the pre-polymer which is leached out after curing.

Batches of pre-polymer were prepared according to the design matrix. A Pre-polymer was injected into a polydimethylsiloxane mold before the curing step, where each sample was then individually exposed to the experiment-prescribed UV exposure energy dose. Samples were designed as thin strips (length: 10 mm, width: 1.5 mm, thickness: 0.1 mm) according to an ASTM D882 standard modified to be compatible with the equipment available in the laboratory for conducting tensile tests. Samples were collected and soaked in PBS for a minimum of 24 hours before tensile testing. Tensile tests were conducted in PBS with a Myograph (KENT SCIENTIFIC®). Elasticity was taken as the slope of the linear portion of the generated stress-strain curve and the test was carried out to failure.

### Transwell Assay for Polymer Degradation

Pre-polymer strips (124, POMaC, 124+40% porogen, POMaC+40% porogen) (1.5 mm×0.5 mm×10 mm) were exposed to UV (365 nm) energy. The strips were weighed in sets of 8 to determine the initial mass. Strips were soaked in PBS for 2 hours, 70% ethanol overnight, and washed twice with sterile PBS. These were then placed in base of transwell insert 24-well plates (Corning), with rat-CM seeded in the transwell inserts and cultivated in rat CM media. The strips were collected at days 1 and 14, washed twice in deionized distilled water and dried under lyophilization for two days. Final mass was measured, recorded, and expressed as a percentage loss of the initial dry mass (day 0). Strips were also characterization for a change in mechanical properties, as described in mechanical characterization.

### Hydrolytic Degradation

UV cured polymer strips, as described in transwell degradation, were placed in pre-weighed 20 mL glass scintillation vials in sets of 8 and the initial dry mass was recorded. 10 mL of PBS was added to each vial, each was sealed and placed in 37° C. environment under agitation. Vials were collected at days 1, 7, and 30, dried under lyophilization for 2 days and the final dry mass was recorded. Degradation was reported as percentage of initial mass lost.

### Cell Seeding on Mesh Patches

Scaffold meshes were first coated with 0.2% (wt) gelatin in PBS (37° C.) for 3 hours to assist in cell attachment. Freshly isolated rat CMs were first pelleted and suspended in liquid Matrigel solution (1 million cells/1 uL Matrigel). Two microliters of cell suspension were pipetted onto the mesh surface, placed in a well of a 6 well plate (1 mesh/well). Excess cell was removed to ensure only a thin layer of cell solution covered each mesh. The well plates with the scaffolds were incubated (37° C., 4-6 min) to allow for partial gelation. Rat culture media (3 mL/well, 37° C.) was then added to the plates and mesh scaffolds were gently scraped from the bottom of the plate and allowed to float. Cell culture media was changed every 48 hours. Immuno-fluorescent staining was performed by first fixing the tissues in 4% (w/v) paraformaldehyde in PBS for 15 min at room temperature. Then, the cells were permeated and blocked in 10% FBS and 0.25% Triton X100 in PBS for 1 hour. Next, the tissues were incubated in primary antibody against Troponin T (Mouse, 1:200), overnight at 4° C., followed by incubation with a secondary antibody, TRITC anti-mouse IgG and a phalliodin 66 conjugated anti-F-actin. Tissues were then imaged with confocal microscopy (OLYMPUS® FV5-PSU confocal with IX70 microscope, Canada).

### In Vivo Study

The 124 polymer and poly (L-lactic acid) (PLLA) (as relative control) discs (8 mm (d)×1.5 mm (t)) were used to assess in vivo host response. 124 polymer discs were prepared from pre-polymer with a 1:4 ratio of 124-butanetricarboxylic acid to maleic anhydride and cured with 54000 mJ/cm² of UV energy. PLLA (SIGMA®) discs were cast in chloroform (SIGMA®) and the solvent was removed. Discs (n=5) were sterilized in 70% ethanol and washed twice in sterile PBS. Discs were implanted subcutaneously in the back of Lewis rats (CHARLES RIVER®). Implantation side and order was randomized to ensure independence. Rats were euthanized 60 days post-implantation, tissue surrounding the discs was excised and fixed in 10% formalin for 24h. Samples were then placed in PBS and sent for paraffin-embedding and sectioning at the Pathology Research Program (University Health Network, Toronto, ON). Slides were stained with Masson's trichrome, CD68, CD163, CD3, CD31, and smooth muscle action (SMA). Cell presence was assessed as an average of five samples percentage of total area (400 pixels from implant edge) with positive stain.

### Results and Discussion

### Rationale for Monomer Selection and the Approach to Synthesis

Development of the elastomeric material for cardiac tissue engineering relied on four design criteria: (a) A simple synthesis procedure that utilizes monomers that are both cost-effective and regarded as safe in their singular form; (b) possession of highly tunable mechanical properties which fall within the range of human cardiac tissue; (c) the ability to serve as a scaffold material for cardiac cells in vitro; and (d) the presentation of minimal host response with subcutaneous implantation.

The previously described POMaC material provides many of the properties desired for functional tissue engineering applications; however it is limited by its elasticity for cardiac tissue engineering use (Elongation: 194%, Young's modulus: 0.29MPa, Tensile Strength: 611 kPa) (US patent reference). With this in mind, a novel material with improved elastomeric properties for the purpose of cardiac tissue engineering is desired.

Here, a novel polyester biomaterial, 124 polymer, that exhibits improved elastic properties for cardiac tissue engineering applications is described. By substitute citric acid with 1,2,4-butanetricarboxylic acid, the 124 polymer substitutes the four-armed polymer network in POMaC with a three-armed polymer network, which, in turn, lower the Young's modulus for cardiac applications.

In order to prepare an improved polyester material, it was necessary to react an alcohol with an acid. Thus, a list of diol and carboxylic acid candidates were evaluated for potential synthesis. Polymerization of tartaric acid and malic acid (in two separate trials) in a copolymer with 1,8 octanediol and maleic anhydride were performed. Both of these results proved unsuccessful: tartaric acid polymer exhibited cytotoxic effects when exposed to rat cardiac fibroblasts, which was attributed to its properties as a Kreb's cycle inhibitor; and malic acid polymer was unable to form sufficient crosslinks to serve as an elastomeric material.

These findings suggested it was necessary to maintain the uneven ratio of functional groups of monomers e.g., 2:3, in order to enable branching and crosslinking of linear polymer chains to form a three dimensional polymer network. However, excessive branching may also increase the stiffness and decrease the elasticity of a polymer network. Thus a list of diol candidates that would be reacted with candidate tri-carboxylic acids was selected. This lead to the synthesis of the 124 polymer, a tricarboxylic-acid monomer reacted with a diol candidate. Alternatively, triols could be reacted with di-carboxylic acids.

### Characterization of 1,2,4 Polymer (or “124 Polymer”)

Polycondensation of 1,2,4-butanetricarboxylic acid, maleic anhydride and 1,8-octanediol under nitrogen conditions yielded a viscous yellow pre-polymer gel as illustrated in FIG. 1A. In this instance, polymers were reacted in a 2:3 molar ratio of 1,2,4-butanetricarboxylic acid to maleic anhydride, while maintaining an equal number of carboxylic acid to hydroxyl reaction sites. The water-in-air contact angle of pre-polymer was assessed as 84.5°, suggesting relatively hydrophobic surface properties in comparison to other common polyester biomaterials. The relatively low viscosity allows for pre-polymer injection through typical needle gauges, allowing for molding into micro fabricated structures prior to UV exposure.

The structure of this polymer gel was verified with resonance of hydrogen atoms. FIG. 1B presents a representative ¹H-NMR spectrum for 124 pre-polymer. Peak assignment was conducted with respect to tetramethylsilane (TMS). The peaks (1) between 6 and 7 ppm were assigned to the —CH═CH— bonds incorporated into the polymer backbone. The peaks (2) in the range of 3.75-4.5ppm are assigned to —O—CH₂ in the 1,8-octanediol portion of the backbone. Peaks (3,4) (1.27, 1.39,1.58ppm) are assigned to the CH₂—CH₂ bonds from 1,8-octanediol and the variation in shift is attributed to the proximity to the ester bond. The peaks (6-9) at 1.79 ppm and 2.32-2.74 ppm are attributed to the CH₂—CH₂ and the CH₂ bonded to ester or carboxylic acid groups. The high number of peaks in this structure is attributed to the variation in random polymer structure and degree of crosslinking, causing slight shifts in peak location.

### Elastic Modulus Testing and the “Fine-Tuning” of the Elastomer

Using a statistical design of experiments, the relationship between UV exposure energy, monomer feed ratio, and porogen content on the Young's Modulus of photocrosslinked 124 polymer was investigated. For ease of utilizing a design of experiment (DOE) design, the variables were coded according to a general equation, where x is an arbitrary variable (Equation 1).

$${x}_{coded} = \frac{{x}_{uncoded} - {\overline{x}}_{uncoded}}{\frac{1}{2} range ( {x}_{uncoded} )} \qquad ( 1 )$$

This general equation was used to code the high and low values (Equations 2-4), and the associated values are summarized in Table 1.

$$\begin{matrix}A = \frac{( a - 0.875 )}{0.625} & ( 2 ) \\ B = \frac{( b - 18000 mJ )}{9000 mJ} & ( 3 ) \\ C = \frac{( c - 20 \% )}{20 \%} & ( 4 )\end{matrix}$$

TABLE 1
Summary of maximum and minimum values
for the experimental design.

| | Monomer Ratio | UV Exposure | Porogen |
|---|---|---|---|
| Coded Value | (124:MA) | Energy (mJ) | Content (%) |
| −1 | 0.25 | 9000 | 0 |
| 1 | 1.5 | 27000 | 40 |
| 0 | 0.875 | 18000 | 20 |

The factors were varied at high and low levels yielding 2³=8 samples, plus replicates at a midpoint to give a total of 10 samples in the experiment, as outlined in Table 2.

TABLE 2
Design matrix for 1,2,4-polymer's 23
factorial experiment with two midpoints

| | Monomer Ratio | UV Exposure | Porogen |
|---|---|---|---|
| Sample | (124:MA) | Energy (mJ) | Content (%) |
| 1 | 1 | 1 | 1 |
| 2 | −1 | 1 | 1 |
| 3 | −1 | −1 | 1 |
| 4 | 1 | −1 | 1 |
| 5 | 1 | 1 | −1 |
| 6 | −1 | 1 | −1 |
| 7 | 1 | −1 | −1 |
| 8 | −1 | −1 | −1 |
| Replicates | −0.333 | 0 | 0 |

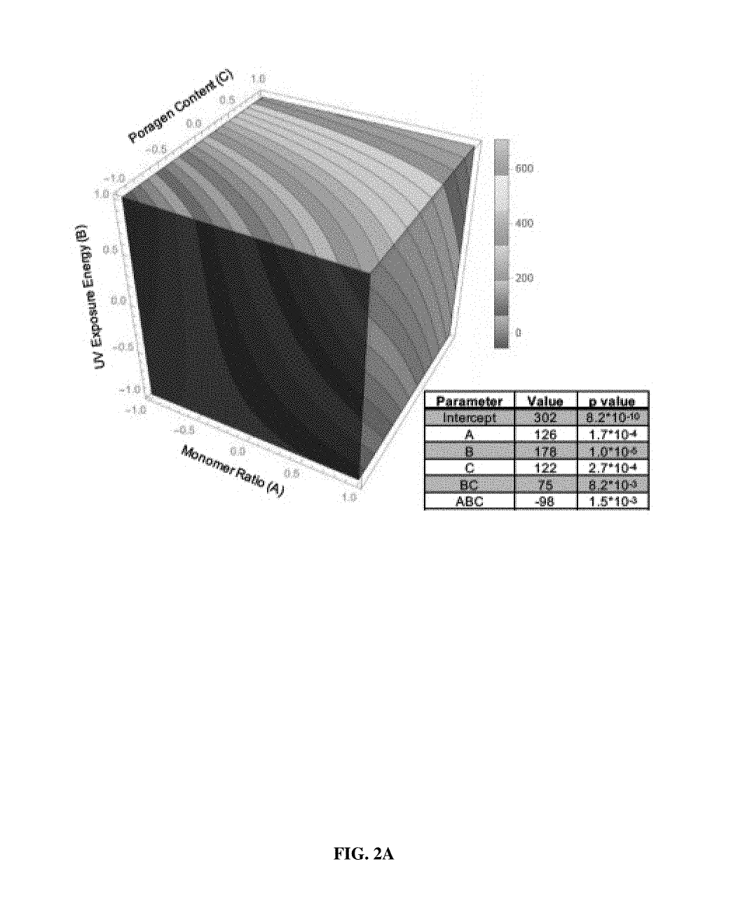

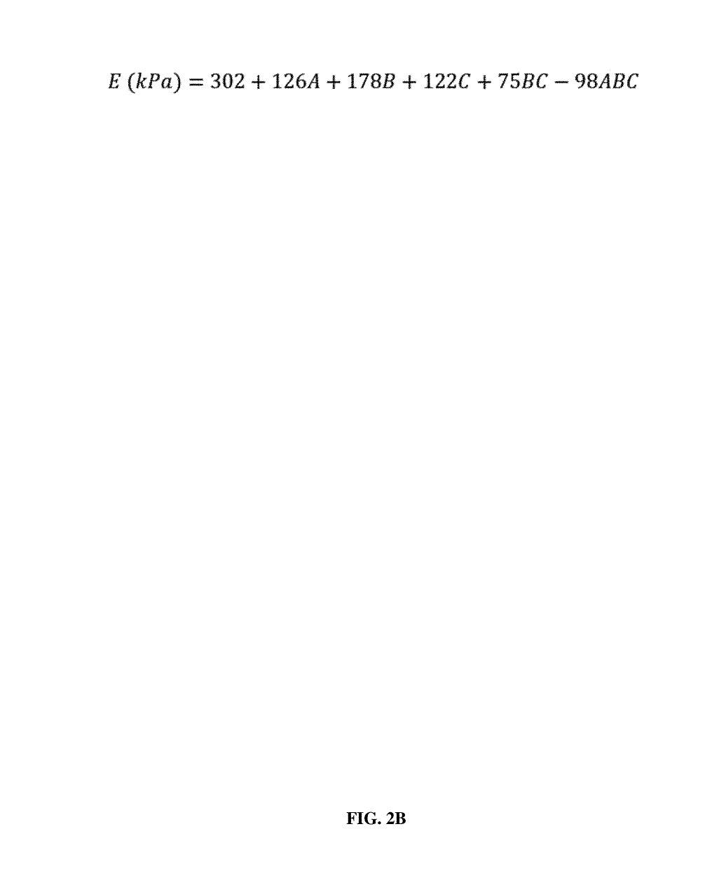

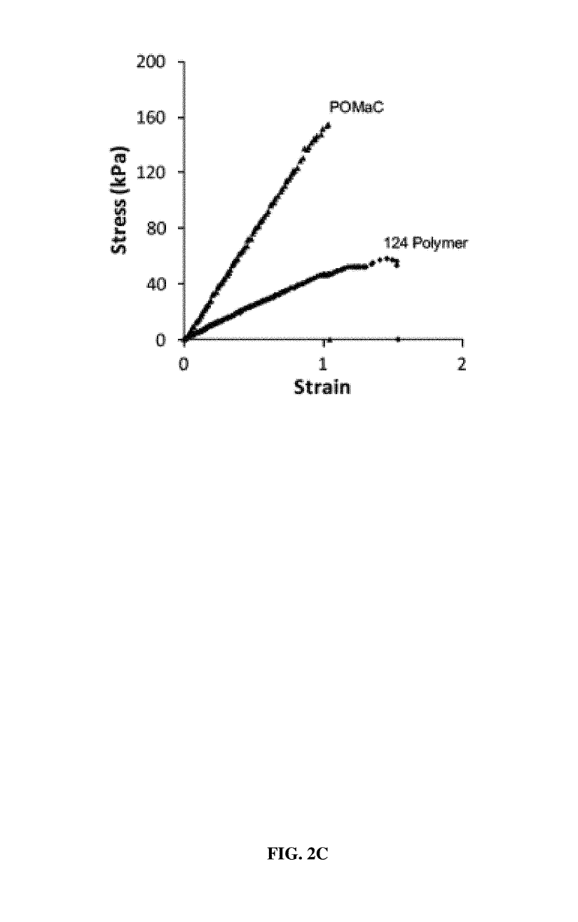

FIG. 2A shows a graphical and mathematical representation of this relationship, and the associated coded variables for this study can be found in Tables 1 and 2. Statistical assessment (p<0.01) was used to remove interaction factors for monomer ratio with UV exposure energy and porogen content. This experimental design developed a mathematical model relating Young's Modulus (E) to the independent factors and their interactions as follows (Equation 5, also as FIG. 2B):

E(kPa)=302+126A+178B+122C+75BC−98ABC (5)

where A, B, and C are coded variables for monomer ratio, UV exposure energy (mJ) and porogen content (%) respectively. This model has increased accuracy with modulus values greater than 100 kPa, as testing at the low extremes of UV exposure energy (−1) and monomer ratio (−1) utilized a material with fluid-like properties.

A positive relationship was observed between each of the individual factors and the modulus of the cross-linked material. This supports the associated chemical theory and hypothesis in designing suitable materials for cardiac applications.

The increase in 1,2,4-butanecarboxylic acid content over maleic anhydride increases the branched structure of the material, and in turn the viscosity and molecular weight of the pre-polymer material. This increase in branched networks decreases the ease at which polymer chains can slide past each other in the polymer bulk, leading to increased stiffness. Increased UV exposure causes further photo-crosslinking, similarly decreasing elastic properties. Thirdly, and as supported by the statistically significant interaction factor between UV exposure and porogen content, the addition of PEG-DM localizes polymer crosslinks. It was noted that while the addition of porogen to pre-polymer generates a miscible polymer blend, it also gives localized pockets of 124 polymer material. Therefore, the effect of UV energy is intensified with additional porogen, providing a final material with a higher degree of crosslinking.

Based on the results of this modeling, a monomer ratio of 2:3 and 1:4 (acid:anhydride) were the focus of the remainder of analysis. The development of this mathematical model allows for the fine-tuning of the final polymer material for applications based on the desired scaffold elasticity. In cardiac tissue engineered solutions this adaptability is highly desired, as there is a link between cellular behavior and the mechanical properties of their surroundings. The values suggest a high degree of elasticity of this material, falling within the range of typical human cardiac properties. Furthermore, when compared to a POMaC control synthesized under comparable conditions, 124 polymer presented a lower Young's Modulus (FIG. 2C), suggesting improved elastomeric characteristics. This is advantageous in cardiac tissue engineering applications, as scaffolds undergo repetitive contractions in support of a beating engineered cardiac tissue.

### Degradation

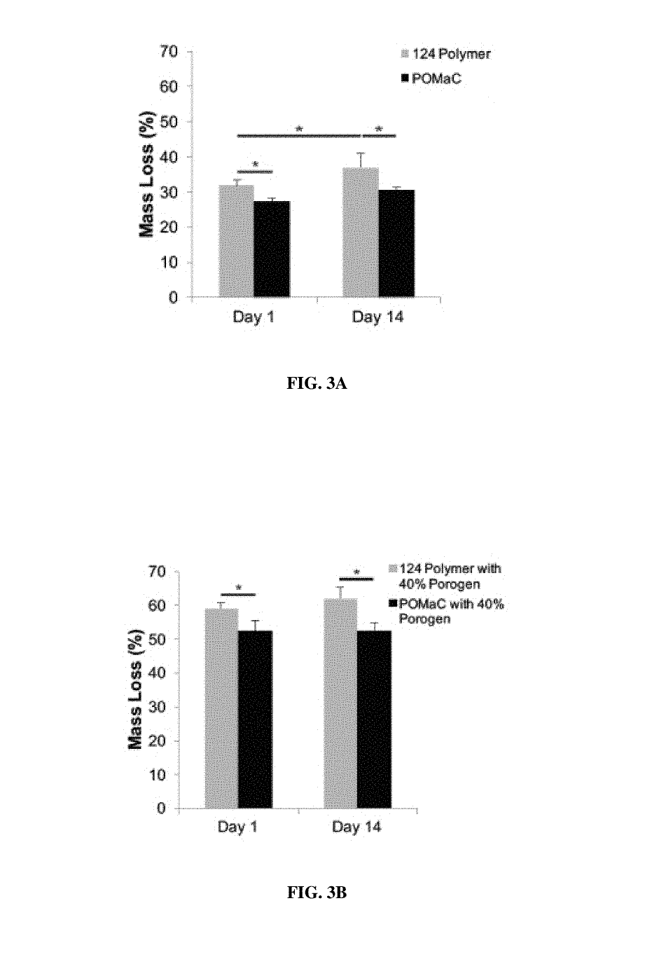

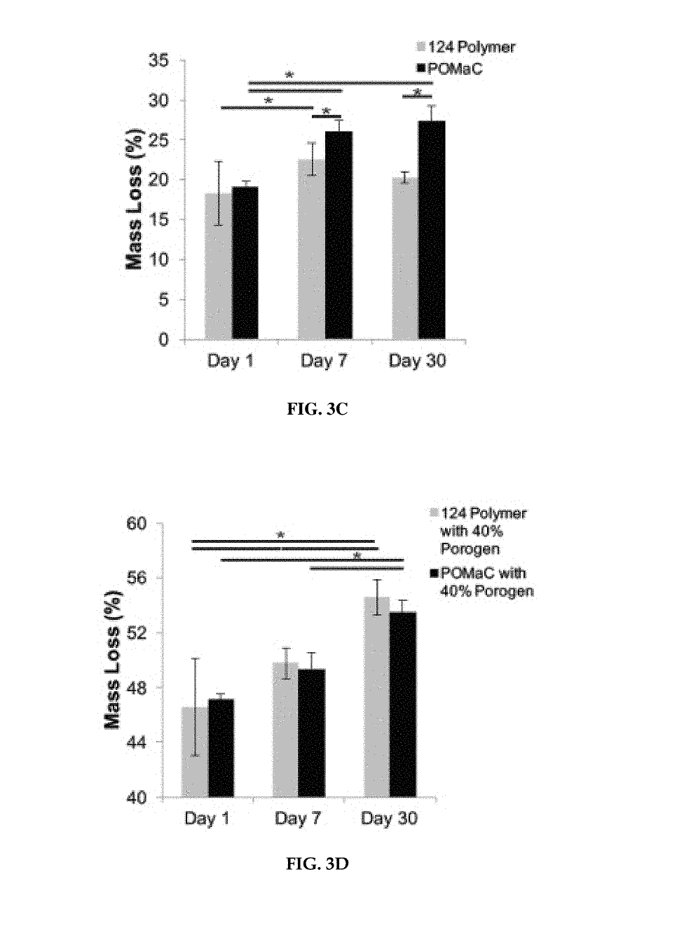

The mass loss and change in Young's Modulus were assessed for photo-crosslinked 124 polymer samples both in phosphate-buffered saline and in transwell plates containing neonatal rat cardiomyocytes. This investigation further assessed the role of porosity, as degradation experiments were conducted with replicates with and without porogen content. The assessment utilized POMaC elastomer as a control, synthesized and UV exposed under similar conditions. The mass loss was assessed over 14 days in transwell conditions (FIGS. 3A and 3B) and in PBS (FIGS. 3C and 3D).

In this degradation assessment, the effects of both hydrolytic degradation mechanisms and potential differences when exposed to the enzymatic environment of cardiac cells were evaluated. Over a 14 day period in a cellular environment, pure 124 polymer exhibited a slight increase in mass loss, which was of statistical significance but did not result in a large decrease in mass (FIG. 3A). Similar results were observed when testing nano-porous material (FIG. 3B). In both situations the mass loss was greater than that of POMaC control (p<0.05), but the magnitude of the difference was small. POMaC showed no appreciable mass loss over 14 days. The initial mass loss in pure materials is attributed to the soluble characteristics of low molecular weight chains of each material, and the additional mass loss of porogen containing materials is attributed to the leaching of water soluble PEGDM.

Under hydrolytic degradation conditions in PBS, mass loss was non-significant in pure 124 polymer over 30 days, which contrasts greater degradation of statistical significance in POMaC control over the same period (FIG. 3C). In samples with initial porogen content hydrolytic degradation was more evident, with significant change in mass loss in each material over 30 day period (FIG. 3D). In these instances there was no difference between the 124 polymer and POMaC control. Similar to the transwell study, mass loss at 1 day is attributed to solubility and porogen leaching when appropriate. Under hydrolytic degradation conditions, the porogen content appears to effect the rate of degradation. This suggests the porous structure allows for improved water penetration into the polymer bulk in both materials, causing appreciable mass loss from the polymer bulk. This property seems to play the opposite effect in the pure material. 124 polymer possesses a more hydrophobic polymer backbone on a molecular level. The limited degradation rate in comparison to the POMaC control could suggest diffusion limitations of water into the polymer bulk. Overall, 124 polymer degradation is relatively similar to that of POMaC control, and shows the ability to maintain structure over a 1 month period, which supports its applicability in tissue engineering constructs. Comparison between degradation samples in contact with cells to those that were not in contact with the cells shows a noticeably higher mass loss of 124 polymer, particularly over the first 24 hours. This suggests the solution properties of rat cardiomyocyte growth media and ethanol sterilization may have increased the solubility of low molecular weight polymer chains.

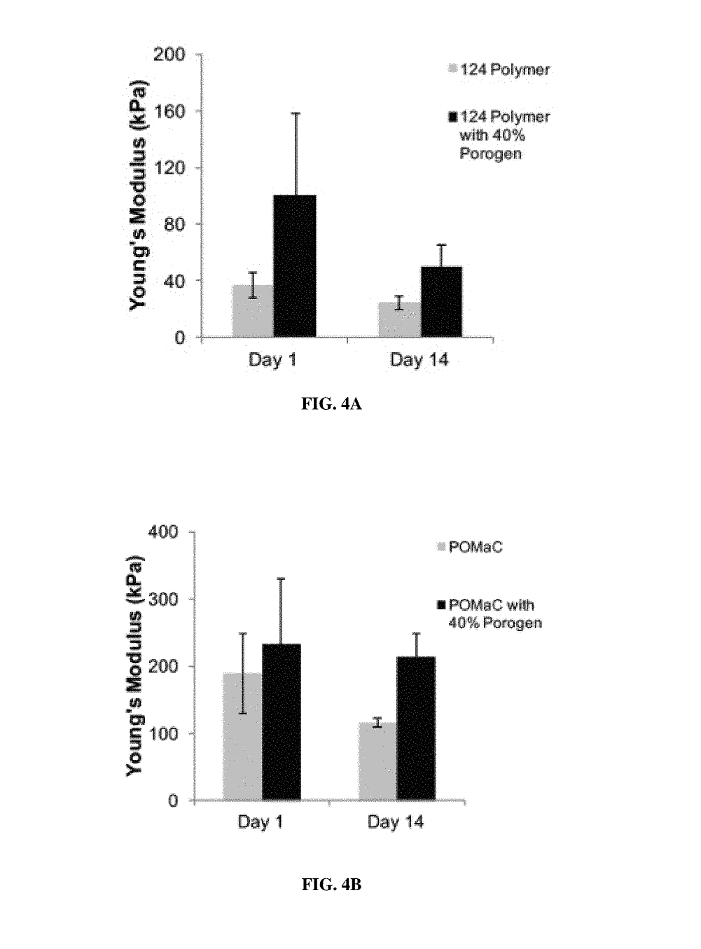

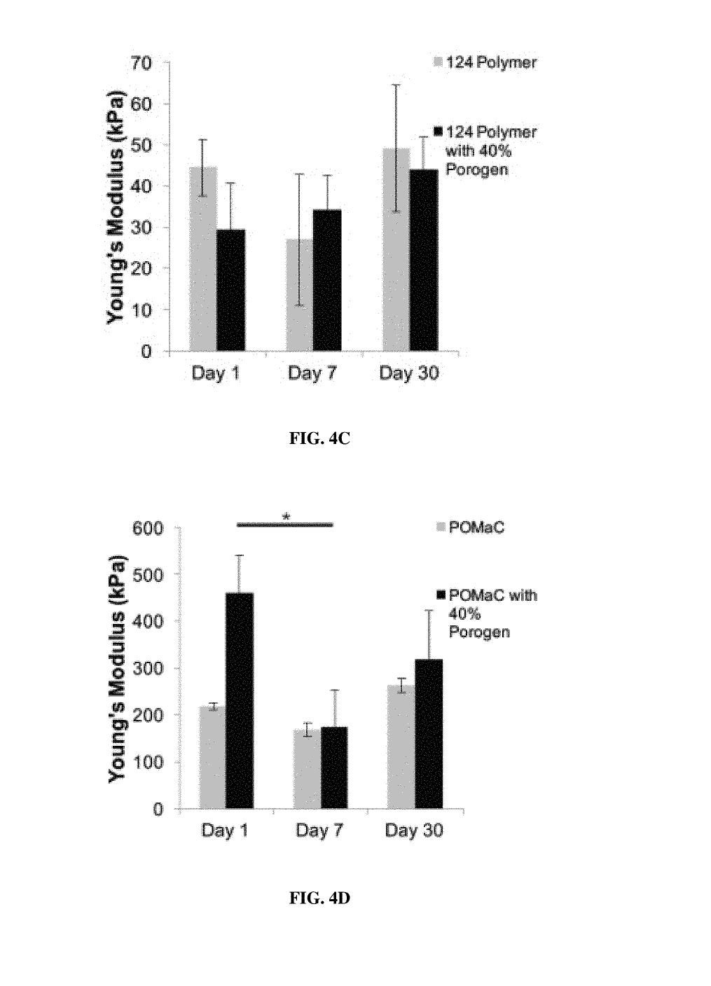

Co-current assessment was conducted on the change of Young's Modulus as a measure of potential bulk erosion (FIGS. 4A-4D). There was no appreciable decrease in the Young's modulus of the samples tested, suggesting the material maintained mechanical properties over a 14 day and 1 month period for transwell and hydrolytic degradation conditions respectively. This further supports potential applicability in tissue engineered constructs, as mechanical stability is an important aspect to structural support. Furthermore, this study allowed for a direct comparison of elastic properties of 124 polymer to those of POMaC, and 124 polymer exhibited a significantly lower Young's modulus than POMaC, both initially and over time, suggesting 124 polymer is more elastic. This could be attributed to the absence of the hydroxyl pendant group found on citric acid in POMaC, suggesting there may be a greater polymer chain length between entanglements. This improvement is desirable for support of cardiac tissue engineering constructs, as there is less potential for inhibition of contraction in immature tissues.

### In Vitro Cell Attachment

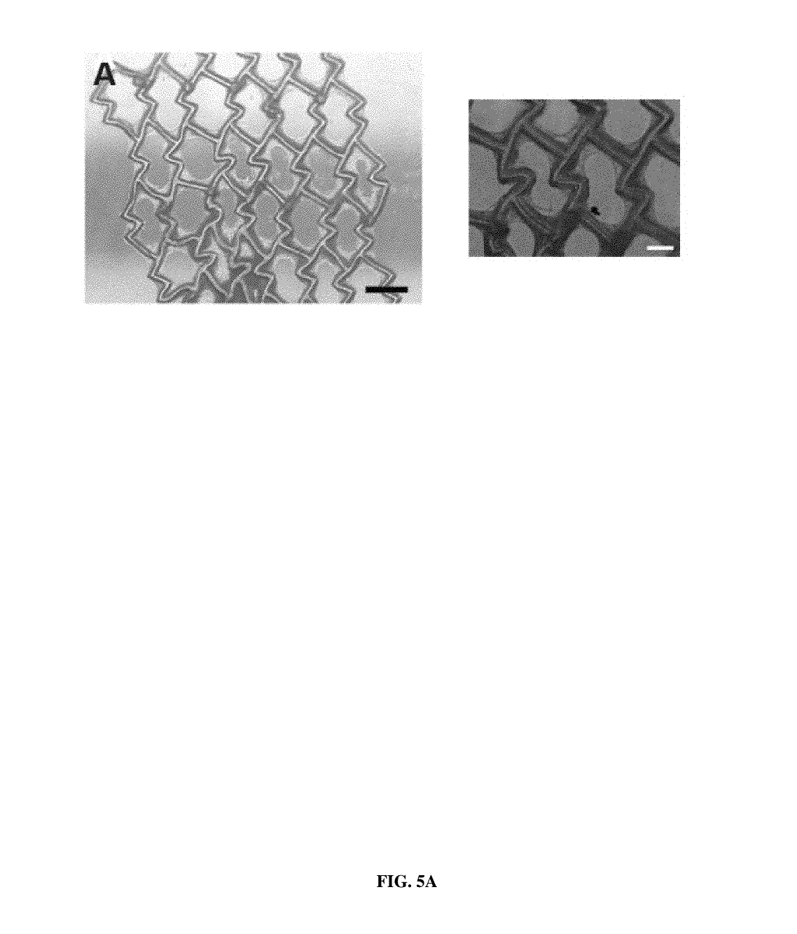

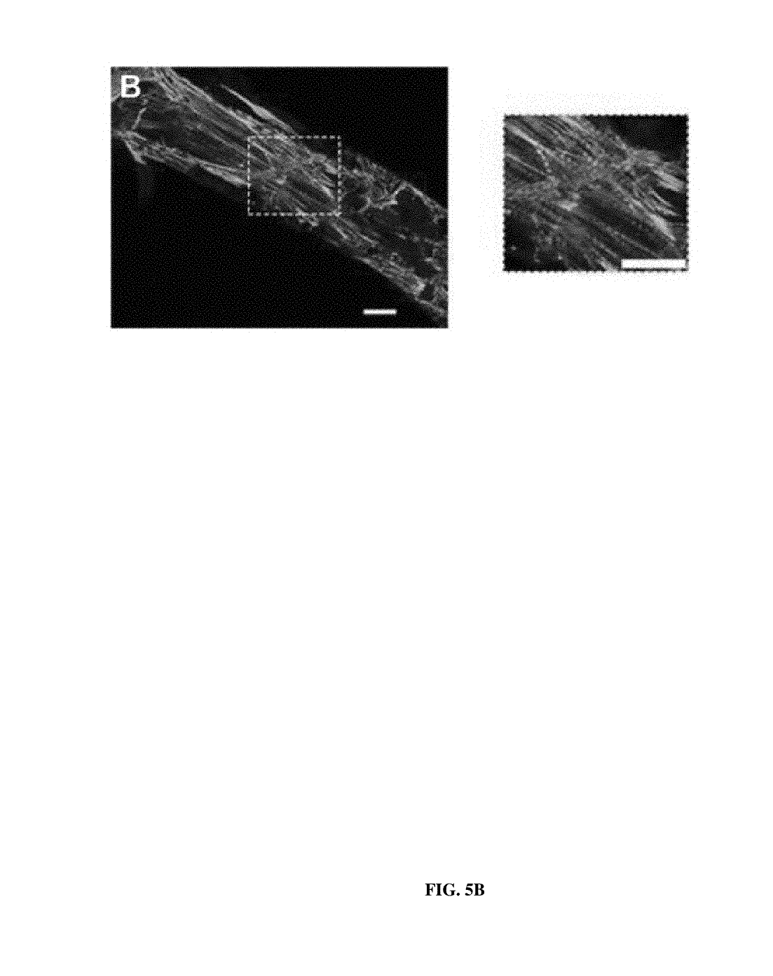

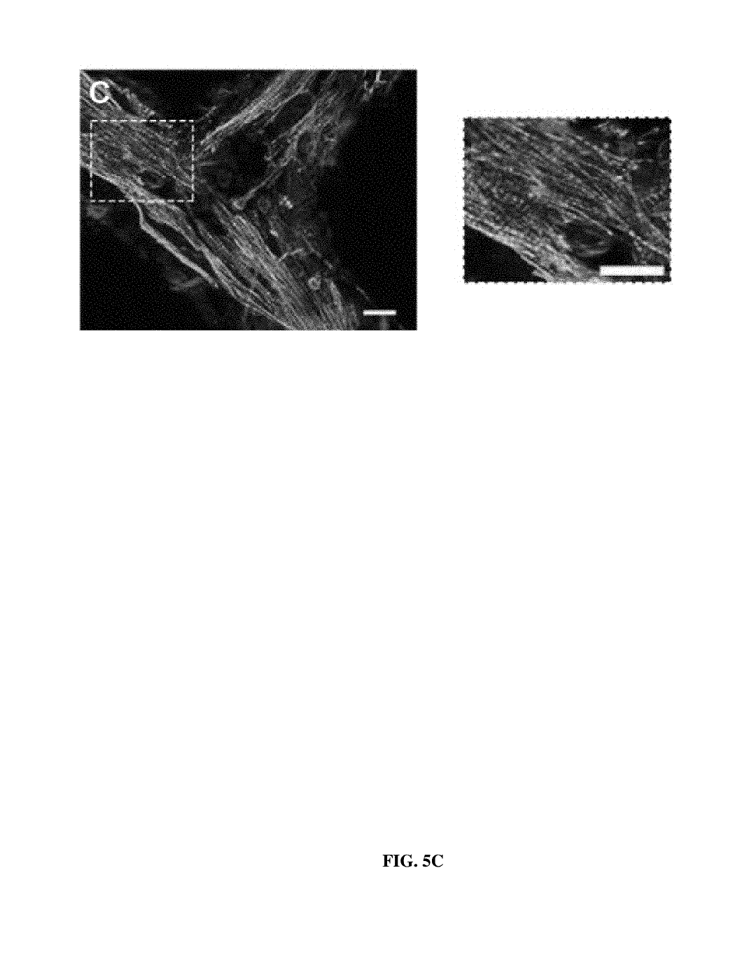

Mesh scaffolds based on previously published design were injected with 124 pre-polymer gel and photo-crosslinked to generate an elastomeric tissue engineering construct. Rat cardiomyocytes seeded on the scaffolds were assessed for cell survival through confocal imaging (FIGS. 5A-5C).

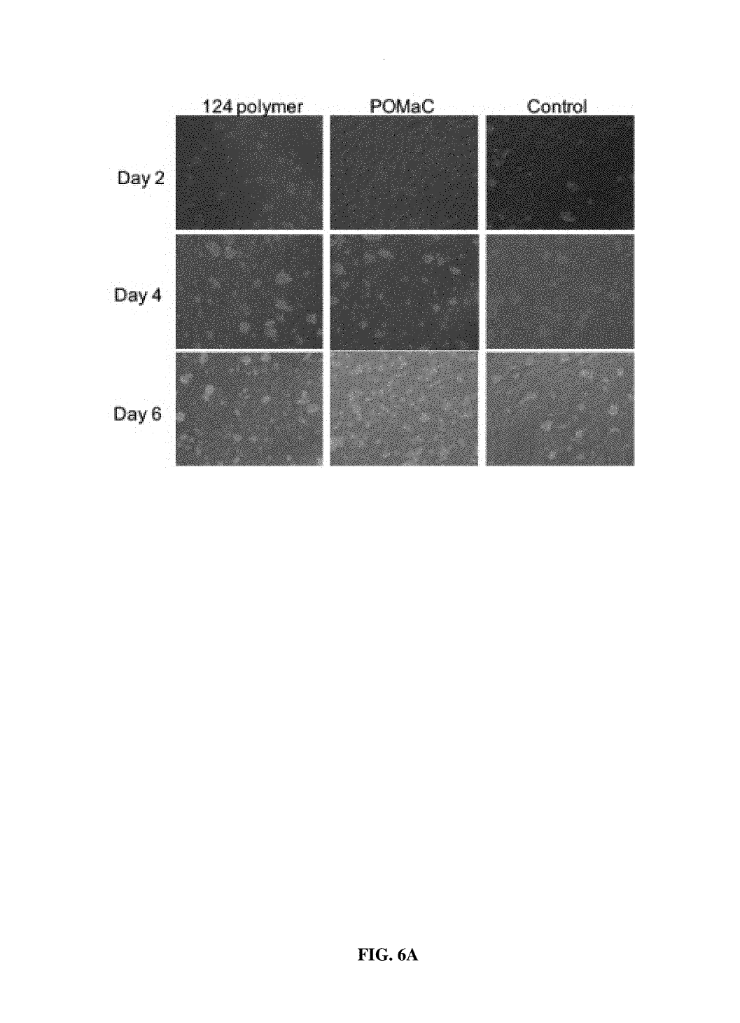

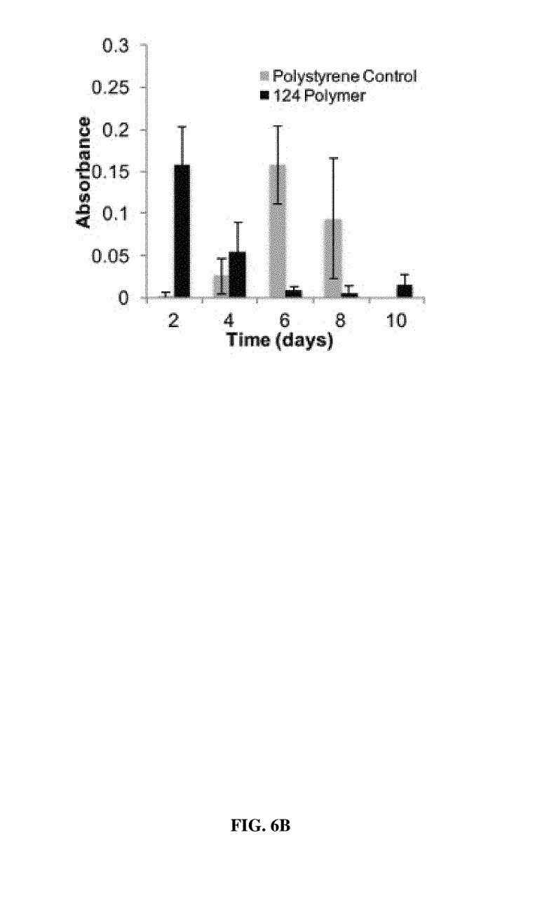

Staining with cardiac troponin-T and F-actin and imaging by confocal microscopy demonstrated the formation and survival of rat cardiomyocyte tissue on the engineered mesh (FIGS. 5B and 5C). Cross-striations were evident (FIGS. 5B and 5C), further supporting the presence of an organized cardiac tissue. Corresponding bright-field imaging of these scaffolds shows tissue development around the accordion-like mesh repeating unit. This tissue demonstrated synchronous beating under electrical stimulation seven days post seeding. Real-time observation shows evident compression of the accordion scaffold design, suggesting the elastic properties of the material support the synchronous tissue contraction. This analysis suggests the applicability of crosslinked 124 polymer as a tissue engineering scaffold, as it supports cell survival and tissue development in vitro. Non-cytotoxic effects are further observed with no significant difference in cell death in longer-term culture in monolayer cardiac fibroblast growth against a polystyrene control as shown in FIG. 6A. Furthermore, the construction of these microfabricated structures with 124 polymer demonstrates the ability to mold this polymer into intricate shapes. As the material is fairly non-viscous and can be easily injected, molds of scaffolds such as those shown here on the 1 mm scale are constructed without solvent. This is an important feature for construction of solvent-free tissue engineering scaffolds with complex micro-scale features.

### In Vivo Host Response

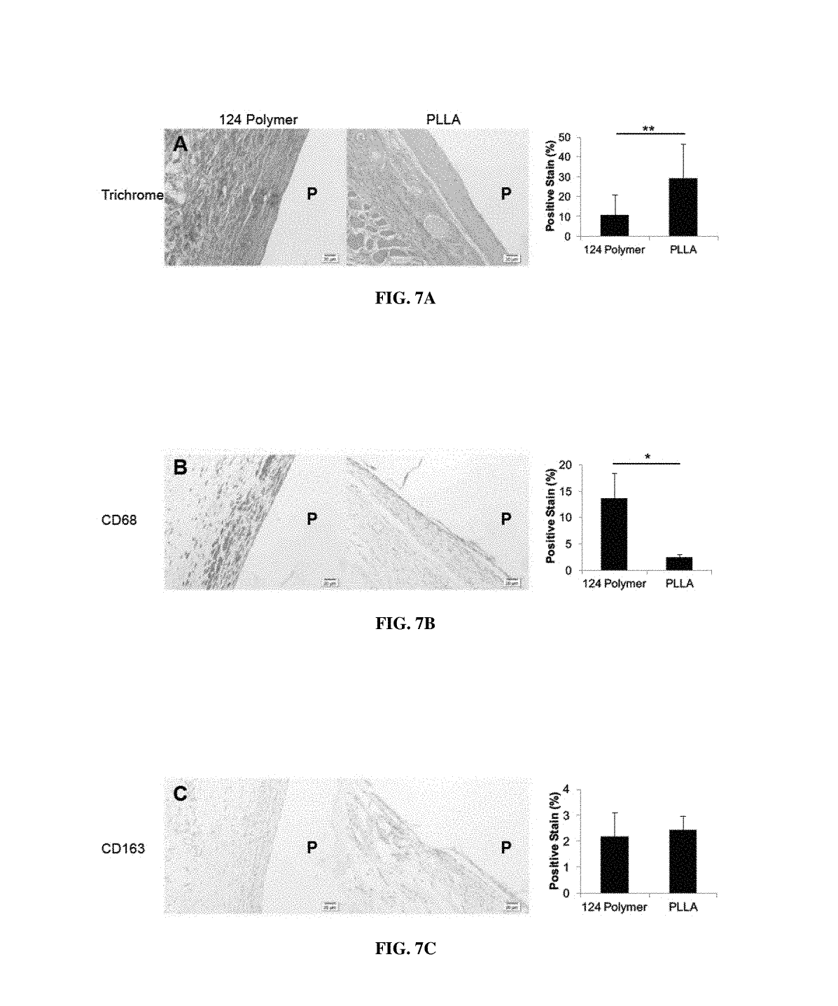

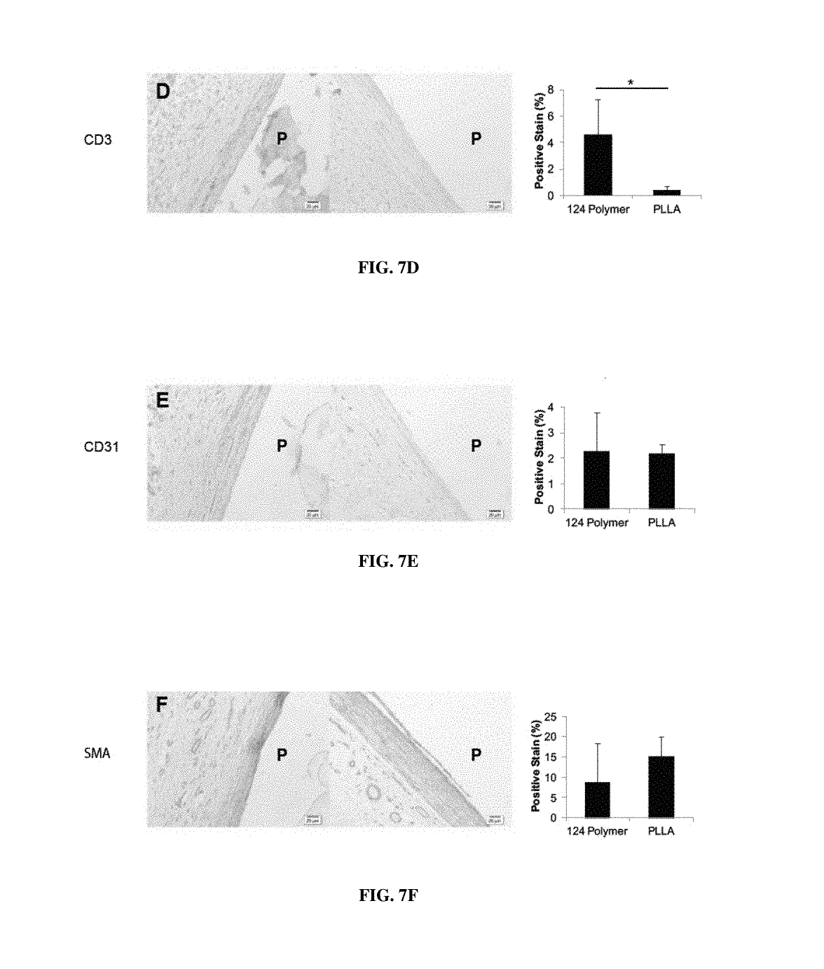

The in vivo host response to 124 polymer was assessed with subcutaneous implantation of crosslinked polymer discs (n=5) as well as PLLA control discs (n=5) over a 60 day period. FIGS. 7A-7F show photomicrographs of slides stained with specific markers. Comparison of 124 polymer to PLLA in vivo allows for assessment of relative biocompatibility of the new material, as PLLA is generally regarded as a safe implant material. Quantification of positive stain area (%) gives insight into the quantitative comparison of these two materials.

A significant difference is seen in the two materials for Masson's Trichrome, CD68 and CD3 staining (FIGS. 7A, 7B, and 7D). Masson's Trichrome was quantified for the percentage of blue staining, which is associated with collagen content. CD3 is a marker for T-cell immune recruitment, and CD68 is associated with total macrophage cell number. Quantification shows an increased T-cell and macrophage recruitment along the implant boundary and a decreased collagen content in 124 polymer samples in comparison to the PLLA control. Most likely, this can be attributed to a delayed host response, in 124 polymer compared to the PLLA control. The deposition of dense collagen is an effort to segregate the implant, a final effort by the host to defend against the foreign object. Although we see an increased macrophage presence, we suggest the decreased collagen deposition signifies a less severe host response. Furthermore, although T-cell recruitment is observed, it is in low amounts in both groups. There was no appreciable difference observed in vascular cell markers (CD31- a marker of endothelial cells, SMA—a marker for smooth muscle cells and myofibroblasts), which are utilized to quantify vascularization of the surrounding tissue (FIGS. 7E-7F). Additionally, the presence of M2 (pro-healing) macrophages, were also found in similar quantities between the two materials (FIG. 7C). This suggests that the growth of the surrounding tissue post-inflammation is occurring in a similar fashion in the 124 polymer as in the PLLA control.

### Statistical Analysis

The error bars in figures are representative of standard deviation. Analysis was conducted using SigmaPlot 12. Normality and equality of variance was tested and the appropriate test was selected for each data set. Statistical analysis in FIG. 2A was done with a Student's t-test. Analysis in FIG. 3A-3B and FIGS. 5A-5C utilized one-way ANOVA analysis followed by a Tukey-Kramer test. In FIGS. 3C-3D, two-way ANOVA was used followed by Shapiro-Wilk test.

### Summary

Polyester biomaterials are used in tissue engineering as scaffolds for implantation of tissues developed in vitro. An ideal biodegradable elastomer for cardiac tissue engineering exhibits a relatively low Young's modulus, with high elongation and tensile strength. This Example describes a novel polyester biomaterial that exhibits improved elastic properties for cardiac tissue engineering applications. The pre-polymer (aka monomer) poly(octamethylene maleate (anhydride) 1,2,4-butanetricarboxylate) (124 pre-polymer) pre-polymer gel was synthesized in a one-step polycondensation reaction. The pre-polymer was then molded as desired and exposed to ultraviolet (UV) light to produce a crosslinked elastomer, referred to as the 124 polymer. 124 polymer exhibited highly elastic properties under aqueous conditions that were tunable according to the UV light exposure, monomer composition, and porosity of the cured elastomer. Its elastomeric properties fell within the range of adult heart myocardium, but they could also be optimized for higher elasticity for weaker immature constructs. The polymer showed relatively stable degradation characteristics, both hydrolytically and in a cellular environment, suggesting maintenance of material properties as a scaffold support for potential tissue implants. When assessed for cell interaction, this polymer supported rat cardiac cell attachment in vitro as well as decreased fibrous capsule formation in vivo when compared to poly(L-lactic acid) control. Moreover, when compared to a POMaC control, 124 polymer was significantly more elastic (Young's Modulus) in PBS. The improved elastic properties are desired for cardiac tissue engineering applications, as the material could be less inhibitory of cardiac tissue contraction while also providing structural support for the engineered constructs. This polymer showed similar degradation properties to POMaC, both hydrolytically and in a cellular environment. When assessed for cell interaction, this polymer showed the ability for rat cardiac cell attachment as well as decreased fibrous capsule formation when compared to PLLA.

Furthermore, the highly elastic polyester could be molded and photocrosslinked into a complex mesh structure with feature size on the order of tens of micrometers, demonstrating utility in cardiac tissue engineering constructs.

Together these results suggest the potential applicability of this material as an elastomer for cardiac tissue engineered constructs.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims.

1. A pre-polymer having the structure of Formula I for use in forming a crosslinked elastomer for tissue engineering scaffolds: 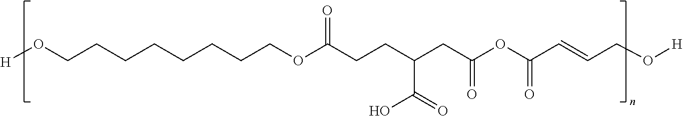
(Formula I), wherein n can be 1 to 1000 units, wherein said pre-polymer is formed by polycondensation of at least one tricarboxylic acid; at least one diol; and at least one unsaturated di-acid.

2. The pre-polymer of claim 1, wherein n is 1 to 100 units.

3. The pre-polymer of claim 1, wherein n is 1 to 50 units.

4. The pre-polymer of claim 1, wherein n is 1 to 10 units.

5. The pre-polymer of claim 1, wherein the tissue engineering scaffold is for cardiac tissue.

6. An elastomer formed by crosslinking two or more pre-polymers of claim 1 to form a network of pre-polymers.

7. The elastomer of claim 6, wherein the crosslinking includes UV crosslinks formed between the unsaturated di-acids of at least two adjacent pre-polymers.

8. The elastomer of claim 6, wherein the crosslinking includes intermolecular ester bonds formed between the tricarboxylic acid moieties of adjacent pre-polymers.

9. The elastomer of claim 6, wherein said elastomer possesses a Young's modulus that is about the same as the Young's modulus of adult cardiomyocte tissue.

10. The pre-polymer of claim 1, wherein the at least one tricarboxylic acid is 1,2,4-butanetricarboxylic acid.

11. The pre-polymer of claim 1, wherein the at least one diol is 1,8-octanediol.

12. The pre-polymer of claim 1, wherein the at least one unsaturated di-acid is maleic anhydride.

13. A three-dimensional tissue engineering scaffold material capable of growing cells, comprising the elastomer of claim 6.

14. The three-dimensional tissue engineering scaffold material, wherein the scaffold is a biowire system, a biotube system, a biorod system, an angiochip system, or an antiotube system.

15. The three-dimensional tissue engineering scaffold material of claim 13, further comprising cardiac tissue growing thereon.

16. The pre-polymer according to claim 1, wherein the polyester has relatively low viscosity for injection through needle gauges.

17. A method of making a dual crosslinkable pre-polymer comprising: combining at one diol, at least one tricarboxylic acid, and at least one unsaturated di-acid to form a reaction mixture, conducting a polycondensation reaction to form the pre-polymer, which is capable of dual crosslinking.

18. A method of making an elastomer comprising: combining at one diol, at least one tricarboxylic acid, and at least one unsaturated di-acid to form a reaction mixture, conducting a polycondensation reaction to form the pre-polymer, which is capable of dual crosslinking; and
exposing the reaction mixture to UV light to introduce UV crosslinks between the di-acid groups of adjacent pre-polymers, and optionally, further optionally introducing one or more ester bonds between tricarboxylic acid groups by polycondensation reaction.

19. The method of claim 17, wherein the ratio of the diol to the tricarboxylic acid is about 0.01 to 1, about 0.05 to 1, about 0.10 to 1, about 0.20 to 1, about 0.30 to 1, about 0.50 to 1, about 0.60 to 1, about 0.70 to 1, about 0.80 to 1, about 0.90 to 1, about 1:1, 1:2, or about 1:3, or about 1:4, or about 1:5, or about 1:6, or about 1:7, or about 1:8, or about 1:9, or about 1:10, or up to about 1:25, or 1:50, or 1:100, or more.

20. The method of claim 17, wherein the ratio of the diol to the unsaturated di-acid is about diol to the unsaturated di-acid can be about 0.01 to 1, about 0.05 to 1, about 0.10 to 1, about 0.20 to 1, about 0.30 to 1, about 0.50 to 1, about 0.60 to 1, about 0.70 to 1, about 0.80 to 1, about 0.90 to 1, about 1:1, 1:2, or about 1:3, or about 1:4, or about 1:5, or about 1:6, or about 1:7, or about 1:8, or about 1:9, or about 1:10, or up to about 1:25, or 1:50, or 1:100, or more.

21. The method of claim 17, wherein the ratio of the tricarboxylic acid to the unsaturated di-acid can be about 0.01 to 1, about 0.05 to 1, about 0.10 to 1, about 0.20 to 1, about 0.30 to 1, about 0.50 to 1, about 0.60 to 1, about 0.70 to 1, about 0.80 to 1, about 0.90 to 1, about 1:1, 1:2, or about 1:3, or about 1:4, or about 1:5, or about 1:6, or about 1:7, or about 1:8, or about 1:9, or about 1:10, or up to about 1:25, or 1:50, or 1:100, or more.

22. A crosslinked elastomer comprising:
polyester consisting of monomers of dicarboxylic acid; at least one triol; and at least one unsaturated di-acid;
wherein the polyester is produced through polycondensation reaction;
wherein the polyester has relatively low viscosity for injection through typical needle gauges; and
wherein the injected polyester was molded into micro fabricated structures and subsequently exposed to UV to form a crosslinked elastomer.

23. A crosslinked elastomer comprising:
polyester consisting of monomers of 1,2,4-butanetricarboxylic acid; at least one diol; and
at least one unsaturated di-acid;
wherein the polyester is produced through polycondensation reaction; and
wherein the polyester is exposed to ultraviolet (UV) light to produce a crosslinked elastomer, and optionally comprises one or more ester bonds formed between monomers of 1,2,4-butanetricarboxylic acid in adjacent polyesters.

24. The crosslinked elastomer according to claims 23, wherein the diol comprises poly ethylene glycol, saturated aliphatic diols, macrodiols, or a mixture thereof.

25. The crosslinked elastomer according to claims 23, wherein the unsaturated di-acid comprises maleic acid, fumaric acid, maleic anhydride, fumaryl chloride, or a mixture thereof.

26. The crosslinked elastomer according to claims 23, wherein the diol comprises 1,8-octanediol and the unsaturated di-acid comprises maleic anhydride.

27. The crosslinked elastomer according to claim 23, further comprises one or more crosslinkers.

28. The crosslinked elastomer according to claims 23, wherein the ratio of hydroxyl to carboxylic acid end groups in monomers is 1:1.

29. The crosslinked elastomer according to claim 23, wherein the ratio of tricarboxylic acid to maleic anhydride groups in monomers is 2:3 or 1:4.

30. The crosslinked elastomer according to claim 23, wherein the monomers is stirred at 150° C. to form the polyester.

31. The crosslinked elastomer according to claim 23, wherein the pre-polymer further comprises porogen to develop nanoporous structure.

32. The crosslinked elastomer according to claim 23, wherein the elastomeric properties match with human cardiac tissue.

33. The crosslinked elastomer according to claim 23, wherein an UV initiator is mixed with the polyester before exposing to ultraviolet (UV) light to produce the crosslinked elastomer.

34. The crosslinked elastomer according to claim 23, wherein the crosslinked elastomer formed scaffold for a soft tissue.

35. The crosslinked elastomer according to claims 23, wherein the crosslinked elastomer formed a cardiac tissue engineered constructs.

36. The crosslinked elastomer according to claims 23, wherein the crosslinked elastomer formed a deformable scaffold element for maturation of human pluripotent stem cell-derived cardiomyocytes.

37. The crosslinked elastomer according to claims 23, wherein the crosslinked elastomer formed a deformable scaffold element for in vitro cardiac cell attachment.

38. The crosslinked elastomer according to claims 23, wherein the formed deformable scaffold element with cardiac cell attachment are used to evaluate the cardiac response to drugs in vitro.

39. A method of evaluating cardiac response to drugs in vitro using crosslinked elastomer comprising:
producing polyester consisting of monomers of 1,2,4-butanetricarboxylic acid; at least one diol; and at least one unsaturated di-acid;
injecting polyester into a mold to form micro fabricated structures;
exposing the molded polyester to UV to form a crosslinked elastomer;
constructing the crosslinked elastomer into a deformable scaffold element;
attaching cardiac cells onto the deformable scaffold element;
exposing the cardiac cells with deformable scaffold element to drugs; and
monitoring the response of cardiac cells through the deformable scaffold element.
